SEQUENCE LISTING (40 sequences)

SEQ ID NO: 1 - DNA, 44 bp, Human
agtaatgatgtcattatccaaacataccttctgtaaaattcatg

SEQ ID NO: 2 - DNA, 28 bp, Human
gtctggaactgcatccaaattcaggttc

SEQ ID NO: 3 - DNA, 13 bp, Human
kmmtgakgtmakm

SEQ ID NO: 4 - DNA, 22 bp, Human
agttaatgattcattatccaaa

SEQ ID NO: 5 - DNA, 14 bp, Human
taatgatgtcatta

SEQ ID NO: 6 - DNA, 25 bp, Human
agttaatgatgtgtcattatccaaa

SEQ ID NO: 7 - DNA, 7 bp, Human
tttggat

SEQ ID NO: 8 - DNA, 23 bp, Human
agttaatgatgtcattatccaaa

SEQ ID NO: 9 - DNA, 14 bp, Human
gaatttggatgcag

SEQ ID NO: 10 - DNA, 14 bp, Human
ctgcatccaaattc

SEQ ID NO: 11 - DNA, 28 bp, Human
gaacctgaatttggatgcagttccagac

SEQ ID NO: 12 - DNA, 14 bp, Human
gtctggaactgcat

SEQ ID NO: 13 - DNA, 14 bp, Human
atgcagttccagac

SEQ ID NO: 14 - DNA, 14 bp, Human
ccaaattcaggttc

SEQ ID NO: 15 - DNA, 14 bp, Human
gaacctgaatttgg

SEQ ID NO: 16 - DNA, 27 bp, Human
gcgaagcttggaagggagagaggttgc

SEQ ID NO: 17 - DNA, 7 bp, Human
atccaaa

SEQ ID NO: 18 - DNA, 16 bp, Human
agtaatgatgtcatta

SEQ ID NO: 19 - DNA, 22 bp, Human
gggccggcgttgttgggcctgg

SEQ ID NO: 20 - DNA, 22 bp, Human
ctgcacaggagggttggaatac

SEQ ID NO: 21 - DNA, 22 bp, Human
ggaatcgtggtctatatccccg

SEQ ID NO: 22 - DNA, 22 bp, Human
tctgcgtcggtaattgaagttg

SEQ ID NO: 23 - DNA, 22 bp, Human
aagccggcatttactcagcccc

SEQ ID NO: 24 - DNA, 22 bp, Human
cgggcagctcggccggctcctc

SEQ ID NO: 25 - DNA, 22 bp, Human
cttgttggagcccacatcgaag

SEQ ID NO: 26 - DNA, 22 bp, Human
ctcttgaatcttcttcatctcc

SEQ ID NO: 27 - DNA, 22 bp, Human
gctgctgctgttgctgctggtg

SEQ ID NO: 28 - DNA, 22 bp, Human
ctgtttgatcaatcttcttccc

SEQ ID NO: 29 - DNA, 22 bp, Human
cggcctcgctgctcatggttgc

SEQ ID NO: 30 - DNA, 22 bp, Human
gccaccgctccctgcgccgctg

SEQ ID NO: 31 - DNA, 22 bp, Human
tgttgatgaaaccatatccgtt

SEQ ID NO: 32 - DNA, 22 bp, Human
ggcagtctggtgtacaaataca

SEQ ID NO: 33 - DNA, 22 bp, Human
taacatttgctgcctccgcacc

SEQ ID NO: 34 - DNA, 22 bp, Human
tctcggtccgccatgatgctgc

SEQ ID NO: 35 - DNA, 22 bp, Human
tcctgcgtctcgtgctgcagcc

SEQ ID NO: 36 - DNA, 22 bp, Human
agagtaaggcggctcttgttgc

SEQ ID NO: 37 - DNA, 22 bp, Human
accaggaactcgcttttgagcg

SEQ ID NO: 38 - DNA, 22 bp, Human
cgggcagcgcaatggtctggcc

SEQ ID NO: 39 - protein, 66 aa, Human
GTVKWFNVRNGYGFINRNDTKEDVFVHQTAIKKNNPRKYLRSVGDGETVEFDVVEGEKGAEAANVT

SEQ ID NO: 40 - protein, 324 aa, Human
MSSEAETQQPPAAPPAAPALSAADTKPGTTGSGAGSGGPGGLTSAAPAGGDKKVIATKVLGTVKWFNVRNGYGFINRNDTKEDVFVHQTAIKKNNPRKYLRSVGDGETVEFDVVEGEKGAEAANVTGPGGVPVQGSKYAADRNHYRRYPRRRGPPRNYQQNYQNSESGEKNEGSESAPEGQAQQRRPYRRRRFPPYYMRRPYGRRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADPPAENSSAPEAEQGGAE

(19) United States
(12) Patent Application Publication
(10) Pub. No.: US 2007/0004666 A1
(43) Pub. Date: Jan. 4, 2007

(54) Methods for modulating apoptotic cell death

(75) Inventors: Annette Lasham, Auckland (NZ); James D. Watson, Auckland (NZ)

Correspondence Address: SPECKMAN LAW GROUP PLLC, 1201 THIRD AVENUE, SUITE 330, SEATTLE, WA, 98101, US

(73) Assignee: Genesis Research & Development Corporation Limited, Parnell (NZ)

(21) Appl. No.: 11/501,319

(22) Filed: Aug. 8, 2006

Related U.S. Application Data
(63) Continuation of application No. 10/028,415, filed on Dec. 20, 2001, now abandoned.
Application No. 10/028,415 is a continuation-in-part of application No. 09/724,809, filed on Nov. 28, 2000, now abandoned.
Application No. 09/724,809 is a continuation-in-part of application No. 09/036,004, filed on Mar. 4, 1998, now abandoned.
Application No. 09/036,004 is a continuation-in-part of application No. 08/713,557, filed on Aug. 30, 1996, now Pat. No. 5,912,168.

(51) Int. Cl. A61K 48/00; C12N 15/09

(52) U.S. Cl. 514/44; 435/455

(57) ABSTRACT

Proteinaceous transcription factors that regulate the transcription of genes and/or the translation of messenger RNA encoding proteins involved in apoptosis, such as CD95 and p53, are disclosed, together with methods for the use of such transcription factors in the modulation of apoptotic cell death. Methods for regulating apoptosis have therapeutic and prophylactic applications for a variety of disorders, including cancer, viral and retroviral infections, neurodegenerative disorders, immune system dysfunction, and other disorders.

### CROSS REFERENCE TO RELATED APPLICATIONS

This application is a continuation of U.S. patent application Ser. No. 10/028,415, filed Dec. 20, 2001; which is a continuation-in-part of U.S. patent application Ser. No. 09/724,809, filed Nov. 28, 2000, now abandoned; which is a continuation-in-part of U.S. patent application Ser. No. 09/036,004, filed Mar. 4, 1998, now abandoned; which is a continuation-in-part of U.S. patent application Ser. No. 08/713,557, filed Aug. 30, 1996, which issued as U.S. Pat. No. 5,912,168.

### FIELD OF THE INVENTION

The present invention relates, generally, to modulation of apoptosis by changes in the level or activity of transcriptional regulators of apoptotic genes. More specifically, the present invention relates to modulation of apoptosis in a population of cells by modulating the cellular level or the activity of a transcription factor polypeptide comprising a member of the Y-box family, such as YB-1, a protein having a cold shock domain, or a homologue of the aforementioned polypeptides.

### BACKGROUND OF THE INVENTION

Apoptosis, or programmed cell death, is a cell suicide mechanism that is used by multicellular organisms to regulate physiological cell death for purposes of defense, development, homeostasis and aging. Regulating apoptosis has important therapeutic and/or prophylactic implications for diseases where apoptosis causes the pathology. Such diseases include chronic neurodegenerative disorders, such as Alzheimer's disease, Parkinson's disease and multiple sclerosis, and immunosuppressive disorders, both genetic and acquired. Moreover, it has been shown that cells exposed to toxins or ischaemia often commit suicide before they are killed by the drug. Modulation of apoptosis may therefore be employed to increase tolerance to pharmaceutical agents, such as chemotherapeutic and radiotherapeutic agents, that stress but that, in the absence of an apoptotic mechanism, may not kill cells. Agents that block apoptosis may also be useful in treating ischemic conditions, such as heart attacks, strokes or reperfusion injury, by blocking the apoptotic response in cells. Pathological suppression of apoptosis appears to be an important factor in neoplastic diseases and viral infection, and may also be involved in infections caused by intracellular pathogens. For example, apoptosis is suppressed in proliferating tumor cells. HIV/AIDS infection produces unregulated and untimely apoptosis in crucial defenders of the immune system, namely CD-4 cells. Regulation of apoptosis may also have implications for in vitro cell growth and maintenance, and may be used to produce more robust cell lines and increase production of recombinant proteins.

Apoptosis is an active process modulated by its own regulatory system and genetics, and is generally characterized by morphological changes including loss of contact of a cell with its neighbors, chromatin condensation, membrane blebbing, cytoplasmic condensation, DNA fragmentation and, eventually, the generation of membrane-enclosed apoptotic bodies that are phagocytosed by neighboring cells. Apoptosis may be divided into four different phases, namely: (1) an initial stimulus which may be either an external or internal signal; (2) detection of this signal and transduction of the signal into the cell; (3) an effector phase which involves the action of proteases; and (4) a postmortem stage during which the cell's DNA is degraded. Many different signal transduction pathways are known to be involved in apoptosis. Apoptosis may be triggered by cell surface receptors of the tumor necrosis factor (TNF) receptor superfamily that transmit extracellular death signals (also know as death receptors) or by cytotoxic agents that inflict cell damage.

One mechanism for the activation of apoptotic cell death is the interaction of the CD95 receptor (also referred to as Fas or APO-1) with its ligand CD95L. CD95 is a member of the TNF receptor family of cell surface proteins, while CD95L is a member of the TNF family of membrane and secreted proteins. CD95 is expressed on a wide variety of cell types, either constitutively or inducibly. For example, it is expressed on activated T and B cells, and its mRNA has been detected in other tissues including thymus, spleen, liver, ovary, lung and heart. CD95 has been implicated in mediating nonspecific T-cell cytotoxicity and activation-induced cell death (AICD) in the peripheral immune system. When apoptosis is induced in T cells by interaction with an antigen receptor, signals are passed into the cell, leading to activation of the cell and expression of c-myc. Both CD95 and CD95L are then up-regulated and expressed on the cell surface. These molecules interact with each other, in an autocrine or paracrine manner, thereby initiating a signaling pathway that induces cell death. Over-expression of the CD95 receptor signaling domain has been shown to result in apoptosis and cell death. A number of tumor cells have very high levels of CD95L mRNA, and negligible levels of CD95 mRNA (e.g. lung, colon, liver and skin carcinomas). These levels are the inverse of those found in non-malignant cells. It is believed that the lack of CD95 expression allows tumors to evade the cytolytic T-cells through the expression of CD95L, thereby inducing apoptosis of the activated T-cells.

Cell damage produced by either chemotherapeutic agents or irradiation typically elicits a DNA damage response, which in turn leads to cell repair, arrest of cell proliferation, or apoptosis. The tumor suppressor p53 plays an important role in signaling cellular responses to DNA damaging agents. See, e.g., Prives, C. and Hall, P. A. (1999). J. Pathology 187: 112-126. In addition, it has been shown that both CD95 and CD95L may be induced after DNA damage by a mechanism that involves p53 (Evan, G. and Littlewood, T. (1998) Science 281: 1317-1321).

In view of the important role of apoptosis in developmental processes, in normal immune function and in the pathogenesis of diverse diseases and conditions, there is an increasing need for novel methods of modulating apoptosis in cell populations and for compositions that may be effectively employed in such methods.

### SUMMARY OF THE INVENTION

In a first aspect, methods are provided for modulating apoptotic cell death in a population of cells, comprising modulating the amount of a transcriptional regulator of apoptosis (TRA) available to bind to a target polynucleotide in the cells. In certain embodiments, the apoptotic cell death is mediated by p53. The cells are preferably selected from the group consisting of tumor cells, cells of the immune system, embryonic cells, cells of the nervous system and cells infected with intracellular pathogens. Preferably the TRA employed in the inventive methods is a member of the Y-box nucleic acid binding family of polypeptides. More preferably the TRA comprises the cold shock domain provided in SEQ ID NO: 39, or a variant thereof, and most preferably the TRA comprises YB-1 (amino acid sequence provided in SEQ ID NO: 40), or a variant thereof.

The amount of TRA available to bind to the target polynucleotide may be reduced by contacting the population of cells with at least one anti-sense oligonucleotide directed against a polynucleotide encoding the TRA, or with at least one decoy oligonucleotide, wherein the decoy oligonucleotide contains a TRA binding site. As described in detail below, the inventors have demonstrated that contacting tumor cells, either in vitro or in vivo, with anti-sense oligonucleotides directed against the TRA YB-1 or with YB-1 decoy oligonucleotides is effective in increasing apoptotic cell death in the cells. The amount of TRA in a population of cells may alternatively be modulated by contacting the population of cells with a genetic construct comprising a polynucleotide encoding a TRA, and suitable promoter and terminator sequences. Examples of YB-1 decoy oligonucleotides include those provided in SEQ ID NO: 2, 9-15 and 17.

In another aspect, methods are provided for modulating apoptosis within a cell population, such methods comprising modulating the binding of a TRA to a regulatory binding site present on a TRA target polynucleotide. Binding of the TRA to its target polynucleotide may be modulated, for example, by contacting the population of cells with a molecule that binds to the TRA binding site.

In yet a further aspect, methods are provided for increasing the sensitivity of tumor cells to a DNA-damaging agent, such as a chemotherapeutic agent. In specific embodiments, such methods comprise contacting the tumor cells with a decoy oligonucleotide comprising a TRA binding site or with an anti-sense oligonucleotide directed against the TRA. Methods for increasing sensitivity to apoptosis in a population of cells harboring intracellular pathogens are also provided, such methods comprising reducing the level of a cold shock protein available to bind to a target polynucleotide in the cells.

Methods for screening for agents that modulate apoptosis are also provided. In one embodiment, such methods comprise providing a population of cells, or a cell extract, that comprises a TRA and at least one TRA binding site, determining the level of free TRA in the cells or cell extract, contacting the cells or cell extract with a candidate apoptosis modulatory agent, and comparing the levels of free TRA before and after treatment. In another embodiment, such methods comprise providing a population of cells that express a TRA and a TRA target gene, incubating the cells in the presence of a candidate apoptosis modulatory agent, and determining whether the level of mRNA transcripts of the TRA target gene is increased or decreased in the cells. In yet a further embodiment, such methods comprise co-transfecting a cell or cell population with (a) a reporter plasmid comprising a regulator polynucleotide from a TRA target gene linked to a promoter and a reporter gene and (b) an expression vector encoding the TRA, and determining the effect of a candidate apoptosis modulatory agent on expression of the reporter gene under conditions where the TRA is over-expressed.

### BRIEF DESCRIPTION OF THE DRAWINGS

Preferred embodiments of the applicants' invention will be described with reference to the drawings, in which:

FIG. 1 illustrates the results of electrophoretic mobility shift assay (EMSA) analysis demonstrating that a hexameric inverted repeat sequence identified in SEQ ID NO: 5 (IR2), present in the hCD95 enhancer region, mediates sequence specific binding of transcription factors in Jurkat cell nuclear extract. Distinct DNA/polypeptide complexes are marked by an arrow and arrowhead. Mutational scanning of the hexameric inverted repeat identified in SEQ ID NO: 5, as shown above the lanes, defined the contributions of individual nucleotide positions to binding and established the degenerate enhancer consensus motif polynucleotide sequence identified in SEQ ID NO: 3.

FIG. 2 illustrates the results of EMSA analysis demonstrating that novel DNA/polypeptide complexes were formed in a sequence-specific manner. Complexes formed with hCD95 enhancer region motifs spaced by 1 bp and 4 bp are marked by an open arrowhead. This data suggests the existence of a family of related transcription factors which recognize the same binding motif but have different spacing requirements.

FIG. 3 illustrates the results of EMSA analysis demonstrating that a novel DNA/polypeptide complex was formed with an hCD95 silencer region probe and an enhancer probe. The experimental work suggested that the polynucleotide heptamer motif identified as SEQ ID NO: 7 or 17 mediates interaction with transcription factor(s).

FIG. 4 illustrates the results of EMSA analysis demonstrating that single-stranded probes compete for complex-formation and interruption of the heptamer motif identified as SEQ ID NO: 7 or 17 in the silencer region abolishes the ability of the probe to compete with wild-type silencer probe for complex formation. The polynucleotide heptamer motif is thus important for regulatory silencing function. The SEQ ID NOS for the probe sequences are identified above the lanes.

FIG. 5 illustrates the results of UV-crosslinking analysis. FIG. 5A shows UV-crosslinking using nuclear extracts from murine L929 cells with a double-stranded hCD95 enhancer region probe (SEQ ID NO: 1). Distinct DNA/polypeptide complexes of approximately 59 and 113 kDA, and a high molecular weight complex of approximately 200-300 kDa are identified. FIG. 5B shows the results of UV-crosslinking using nuclear extracts from Jurkat and L929 cells with a single-stranded hCD95 silencer region probe (SEQ ID NO: 2) to identify DNA/polypeptide complexes of approximately 47, 77 and 100 kDa.

FIG. 6 illustrates the results of Southwestern analysis. FIG. 6A shows the results of Southwestern analysis using nuclear extracts from Jurkat and rat dermal papilla (rDP) cells with a double-stranded hCD95 enhancer region probe (SEQ ID NO: 8). Distinct DNA/polypeptide complexes of approximately 113 kDa (in Jurkat and rDP) and approximately 59 kDa (in rDP) were identified. FIG. 6B shows the results of Southwestern analysis using nuclear extracts from Jurkat cells with a single stranded silencer region probe (SEQ ID NO: 2). Distinct DNA/polypeptide complexes of approximately 47 kDa and 100 kDa were identified.

FIG. 7 shows the changes in expression levels of three YB-1 target genes after treatment with the DNA damaging agent cisplatin. Expression levels of Fas, PCNA and p53 are shown at 0, 2, 4, 6, 14 and 24 hours after cisplatin treatment on a Western blot.

FIG. 8 shows the results of treating SK-MEL-5 cells with increasing amounts of cisplatin-damaged DNA. The number of viable cells after treating the tumor cells with 0. 0.25, 0.5 and 1 μg of cisplatin-damaged DNA was determined and compared to the number of viable cells transfected with untreated (undamaged) DNA.

FIG. 9 shows the results of transfecting SK-Mel-5 cells with DNA treated with increasing amounts of cisplatin. The number of viable cells was determined after treatment of the SK-Mel-5 cells with cisplatin in 3:1, 30:1, 300:1, 3,000:1 and 35,000:1 molar ratios of cisplatin to DNA.

FIG. 10 shows the percentage of THP1 cells expressing MHC Class II after transfection with either YB-1, Mycobacterium tuberculosis cold shock protein, or Mycobacterium bovis cold shock protein. Empty vector (pcDNA3) was used as a negative control.

FIG. 11 shows the binding of T. gondii proteins to the YB-1 binding site of CD95.

### DETAILED DESCRIPTION OF THE INVENTION

In one aspect methods are provided for modulating apoptotic cell death in a population of cells by modulating the level of a transcriptional regulator of apoptosis within the cell population, or by modulating the binding of a transcriptional regulator of apoptosis to a regulatory polynucleotide in the cells. As used herein, the term “transcriptional regulator of apoptosis” refers to a transcription factor that modulates the transcription of a gene, and/or translation of a polypeptide, involved in apoptotic cell death, such as CD95 and/or p53.

Modulation of transcription of genes and/or translation of polypeptides involved in apoptotic cell death may be employed in the therapy of various conditions and disease states by selectively stimulating or inhibiting apoptotic cell death. Disorders that may be effectively treated using the inventive methods include, but are not limited to: conditions characterized by an abnormal or undesired proliferation of cells, such as cancers and other malignancies; neurodegenerative disorders, such as Alzheimer's disease and Parkinson's disease; viral infections, including HIV/AIDS; immunosuppressive disorders; and ischemic conditions, such as stroke, heart attacks, reperfusion injuries. Modulation of the level of a transcriptional regulator of apoptosis in a population of cells may also be employed to modulate the sensitivity of the cells to chemotherapeutic and radiotherapeutic agents. The methods of the present invention may thus be employed in conjunction with chemotherapy or radiotherapy to increase the effectiveness of the therapy and/or to reduce the amount of chemotherapeutic or radiotherapeutic agent required for effective treatment.

In certain embodiments, the transcriptional regulator of apoptosis employed in the inventive methods is a member of the Y-box nucleic acid binding polypeptide family. Preferably, the transcriptional regulator is selected from the group consisting of YB-1, variants of YB-1, and polypeptides comprising a cold shock domain present within YB-1. Most preferably, the transcriptional regulator is selected from the group consisting of human YB-1 (SEQ ID NO: 40, EMBL Accession No. M24070, SWISS-PROT Accession No. P16990), variants of human YB-1 as defined herein, and polypeptides comprising a cold shock domain present within human YB-1 (SEQ ID NO: 39, EMBL Accession No. M24070, SWISS-PROT Accession No. P16990).

YB-1 is a member of a highly conserved nucleic acid-binding polypeptide family known as the Y-box family of proteins. It has been described as a 42-50 kDa protein. Each member of the Y-box family of proteins contains a cold-shock domain which has been identified as a 66 amino acid region (SEQ ID NO: 39), is believed to be a DNA-binding domain, and is highly conserved. These proteins are described, for example, in Wolffe et al. (1992) New Biol. 4:280-298. YB-1 is also known as human dbpB, CCAAT-binding transcription factor 1 subunit A (CBF-A), EF1a or MDR NF-1 (EMBL Accession No. M24070; SWISS-PROT Accession No. P16990); bovine EF1A#1 (EMBL Accession No. M95793; SWISS-PROT Accession No. P16990); rat dbpB, EF1a or CBF-A (EMBL Accession No. M57299; SWISS-PROT Accession No. P22568); murine CBF-A, MUSY1, MSY1, MUSYB (EMBL Accession No. M60419; SWISS-PROT Accession No. P27817), MYB-1A (EMBL Accession No. U33196; SWISS-PROT Accession No. Q60950) or MYB-1B (EMBL Accession No. U33197; SWISS-PROT Accession No. Q60951); rabbit MRNP p50 (EMBL Accession No. U16821; SWISS-PROT Accession No. Q28618); avian EF1a, RSV-EF-1 (EMBL Accession No. L13032; SWISS-PROT Accession No. Q06066), or pYBα (EMBL Accession No. U43513; SWISS-PROT Accession No. Q90376); and frog FRGY1 (EMBL Accession No. M59453; SWISS-PROT Accession No. P21573). YB-1 has been described as a transcription factor for a number of genes associated with cell death, growth and survival in tumor cells, including epidermal growth factor receptor (EGFR) gene, proliferating cell nuclear antigen (PCNA)/cyclin gene, multidrug resistant pump (mdr1) gene and CD-95. In addition, it has also been shown to stimulate transcription of the long terminal repeats (LTRs) of both the human T-cell lymphotropic virus-1 (HTLV-1) and human immunodeficiency virus-1 (HIV-1).

Polypeptides having a cold shock domain that is identical to, or is a variant of, the cold shock domain of human YB-1 (EMBL Accession No. M24070; SWISS-PROT Accession No. P16990) are considered to be members of the Y-box family of binding polypeptides and are especially preferred for use in the methods of the present invention. These include human dbpA (EMBL Accession No. M24069; SWISS-PROT Accession No. P16989); human dbpA-like protein (EMBL Accession No. X95325; SWISS-PROT Accession No. Q14121); human dbpB-like protein (EMBL Accession No. L28809; SWISS-PROT Accession No. P16990); human NSEP-1 (EMBL Accession No. M83234; SWISS-PROT Accession No. Q14972; human nuclease sensitive element binding protein-1 (EMBL Accession No. M85234; SWISS-PROT Accession No. Q15325); rat YB-2 (EMBL Accession No. U22893; SWISS-PROT Accession No. Q62764) and rat RYB-a; murine dbpA (EMBL Accession No. D14485; SWISS-PROT Accession No. Q61478); frog FRGY2, p56, MRNP4 or YB-2 (EMBL Accession No. M59454; SWISS-PROT Accession No. P21574); and frog p54 or MRNP3 (EMBL Accession No. M80257, SWISS-PROT Accession No. P45441). Additional members of the nucleic acid-binding Y-box polypeptide family that have a cold shock domain include proteins from lower organisms, such as and bacterial cspA and cspB. These include, by way of example and without limitation, Salmonella typhimurium CSPA, CSPC, CSPH (SWISS-PROT Accession Nos. P37410, 068636, 033793), Listeria monocytogenes CSPLA, CSPLB (SWISS-PROT Accession Nos. Q48770, P96791), Mycobacterium tuberculosis CSPA (SWISS-PROT Accession No. 006360), Streptococcus pyogenes (SWISS-PROT Accession No. Q54974), Toxoplasma and Chlamydia.

Regulatory polynucleotides that are involved in transcriptional regulation of the CD95 receptor are described in U.S. Pat. No. 5,912,168, the disclosure of which is hereby incorporated by reference. Such polynucleotides are located in a 70bp region about 1 kb upstream from the coding portion of the CD95 gene. Polynucleotides that function as regulatory elements in the enhancement of transcription from the CD95 promoter are described in SEQ ID NO: 1 and 18. The polynucleotide identified as SEQ ID NO: 1 (referred to as E1) exhibits enhancing regulatory activity, but contains both enhancer and silencer regulatory elements. The polynucleotide identified as SEQ ID NO: 18 exhibits enhancing, but not silencing, regulatory activity. A polynucleotide that functions as a regulatory element in silencing transcription from the CD95 promoter (referred to as S1) is described in SEQ ID NO: 2. Both of the polynucleotide sequences described in SEQ ID NO: 1 and 2 provide binding sites for transcription factors that modulate transcription of coding portions of the CD95 gene. A polynucleotide sequence consensus motif that provides a binding site for transcription factor(s) that enhance transcription from the CD95 promoter is set forth in SEQ ID NO: 3. Additional polynucleotides that provide sites for binding of transcription factors that enhance transcription from the CD95 promoter are set forth in SEQ ID NO: 5 and 18, with additional polynucleotides that provide sites for binding of a transcription factor that silences transcription from the CD95 promoter being set forth in SEQ ID NO: 7 and 17.

Transcription of the CD95 gene may be modulated, for example, by blocking the binding of transcription factors to the enhancer or silencer regulatory regions, by modulating expression levels of one or more transcription factors that bind to the regulatory regions, by modulating the binding activity of one or more such transcription factors, or by modulating the functional activity of one or more of such transcription factors. Binding of a transcription factor to an enhancer regulatory region stimulates CD95 expression, while binding of a transcription factor to a silencer regulatory region inhibits CD95 expression. As detailed below, YB-1 binds to both enhancer and silencer regulatory regions of CD95. However, the resulting silencer complexes are considerably more stable than the enhancer complexes. As described below, the inventors have demonstrated that YB-1 down-regulates the expression of CD95, thereby inhibiting apoptotic cell death. Modulation of CD95 gene transcription through modulation of the binding of a Y-box protein to the identified CD95 silencer polynucleotides, renders activated T-cells less susceptible to apoptosis and, at the same time, confers resistance to cytotoxic drugs.

The inventors have further shown that apoptotic cell death can be selectively stimulated in tumor cells by inhibiting expression of YB-1 using anti-sense technology or by providing a decoy oligonucleotide comprising a YB-1 binding site to abrogate the binding of YB-1 to regulatory sequences in the cell's genomic DNA. An unexpected finding was that wild-type, or functional, p53 was required for this response, and that the response was not dependent upon the occurrence of CD95-mediated signaling. The results showed “spontaneous” apoptosis without the addition of any exogenous ligands etc. Reduction of the level of YB-1 available to bind to regulatory polynucleotides involved in apoptosis or blocking of the binding of YB-1 to regulatory polynucleotides involved in apoptosis, may thus be employed to increase the apoptotic susceptibility of tumor cells. Co-transfection of cells with murine p53 promoter fragments linked to a CAT reporter gene and YB-1 showed that YB-1 specifically repressed the p53 promoter. Tumors with either wild-type p53 or mutant p53 capable of restoration of normal function (see, e.g., Chang et al., Molecular Medicine Today (2000), 6:358-364), can thus be selectively targeted for therapeutic intervention, using the methods of the present invention.

YB-1 and the Y-box family of proteins known as cold shock proteins (CSPs) may play an important role in the pathology of disease states other than cancer (e.g., tuberculosis, chlamydial diseases, listeriosis and AIDS-related infections). Bacteria and intracellular pathogens, such as Mycobacterium tuberculosis, Listeria, Salmonella, Chlamydia and Toxoplasma, produce cold shock proteins (CSPs) which have greater than 40% identity to the DNA binding domain of human YB-1, and that can bind specifically to human Y-box sequences and the YB-1 binding site of the human CD95 promoter. See, e.g., Graumann & Marahiel, (1994) FEBS Lett. 338:157-160.

As described below, the inventors have found that the protozoan Toxoplasma gondii encodes in its genome a CSP and expresses a protein that binds to the YB-1 binding site of the human CD95 promoter with the same specificity as YB-1. Several bacterial species and intracellular pathogens have been shown to cause a reduction in the expression of MHC Class II genes (Reiner, (1994) Immunol. Today 15:374-359). In addition, intracellular pathogens such as Chlamydia inhibit apoptosis in infected cells (Fan et al., (1998) J. Exp. Med. 187:487-496). These findings indicate that CSPs produced by bacteria and intracellular pathogens may bind to, and modulate the expression of, cellular YB-1 target polynucleotides of infected cells (e.g., CD95, p53, MHC Class II, and mdr1), thereby protecting the pathogens and their host cells from immune attack and apoptotic cell death. CSPs may also play a role in the development of antibiotic resistance of infected cells. As shown below, the inventors have demonstrated that intracellular pathogens are able to affect host cell immune responses via their cold shock proteins. Thus, reducing the amount of cold shock protein available to bind to a polynucleotide within an infected cell, for example by the use of anti-sense oligonucleotides directed against the cold shock domain of SEQ ID NO: 39 or other regions within the cold shock proteins, decoy oligonucleotides which are bound by the cold shock proteins, or small molecules that interfere with CSP binding to host cell promoters, may be employed to treat infections with intracellular pathogens.

Blocking of the binding of transcription factors, such as YB-1, to regulatory regions of polynucleotides involved in apoptosis, such as CD95 or p53, may be accomplished by introducing small molecules, such as synthetic polyamides, that prevent the binding of transcription factors to regulatory polynucleotides. For example, small molecules may be introduced that target and bind to, or otherwise associate with, the CD95 regulatory sequences identified as SEQ ID NO: 2, 9-15 and 17, thereby inhibiting the binding of transcription factors. The identification and development of suitable small molecules is described, for example, in Cai et al. (1996), Curr. Opin. Biotechnol. 7:608-615 and Gottesfeld et al. (1997), Nature 387:202-205. Blocking of the binding of transcription factors to regulatory regions may additionally or alternatively be accomplished, for example, using oligonucleotide-directed triple helix formation. The use of such methods is documented, for example, in Maher, L. J. (1992), Bioessays 14:807-815 and Chan et al. (1997), J. Mol. Med. 75:267-282.

In certain methods of the present invention, transcription factors are inhibited from binding to regulatory regions of polynucleotides involved in apoptosis by introducing an excess of one or more decoy oligonucleotides, wherein the decoy oligonucleotide comprises a binding site for the transcription factor. For example, transcription factors may be prohibited from binding to CD95 regulatory regions by introducing an excess of polynucleotides comprising the regulatory sequences identified as SEQ ID NO: 2, 9-15 and 17. Excess polynucleotides may be introduced using a variety of techniques well known in the art. Overexpressing a polynucleotide comprising a YB-1 transcription factor binding sequence (such as SEQ ID NO: 2 or 11) in cells, for example, results in binding of endogenous YB-1 to the introduced polynucleotides. Insufficient endogenous YB-1 is then available to bind to the CD95 silencer regulatory region, resulting in activation of the transcription of CD95 and an increase in susceptibility to apoptotic cell death.

Modulating the expression of transcriptional regulators of apoptosis, such as YB-1, may be accomplished by inhibiting translation of the transcription factor. Translation of transcription factors may be inhibited, for example, by introducing anti-sense expression vectors; by introducing anti-sense oligodeoxyribonucleotides, anti-sense phosphorothioate oligodeoxyribonucleotides, anti-sense oligoribonucleotides, or anti-sense phosphorothioate oligoribonucleotides; or by other means well known in the art. All such anti-sense polynucleotides are referred to collectively here as “anti-sense oligonucleotides”.

The anti-sense oligonucleotides disclosed herein are sufficiently complementary to the polynucleotide encoding a transcriptional regulator of apoptosis, such as YB-1, to bind specifically to the polynucleotide. The sequence of an anti-sense oligonucleotide need not be 100% complementary to that of the polynucleotide in order for the anti-sense oligonucleotide to be effective in the inventive methods. Rather an anti-sense oligonucleotide is sufficiently complementary when binding of the anti-sense oligonucleotide to the polynucleotide interferes with the normal function of the polynucleotide to cause a loss of utility, and non-specific binding of the oligonucleotide to other, non-target, sequences is avoided. The present invention thus encompasses polynucleotides in an anti-sense orientation that inhibit translation of the identified transcription factors. The design of appropriate anti-sense oligonucleotides is well known in the art. Exemplary phosphorothioate oligonucleotides designed to the anti-sense strand of YB-1 are identified as SEQ ID NO: 19-23 and 29-33. These polynucleotides are described below with reference to experimental assays.

Cell permeation and activity of anti-sense oligonucleotides can be enhanced by appropriate chemical modifications, such as the use of phenoxazine-substituted C-5 propynyl uracil oligonucleotides (Flanagan et al., (1999) Nat. Biotechnol. 17:48-52) or 2′-O-(2-methoxy) ethyl (2′-MOE)-oligonucleotides (Zhang et al., (2000) Nat. Biotechnol. 18:862-867). The use of techniques involving anti-sense oligonucleotides is well known in the art and is described, for example, in Robinson-Benion et al. (1995), Methods in Enzymol. 254:363-375 and Kawasaki et al. (1996), Artific. Organs 20:836-848. Introduction of an anti-sense oligonucleotide to a CD95 silencer transcription factor, for example, leads to up-regulation of CD95 expression and increases apoptosis in cells that express CD95.

Transcription factors, such as YB-1, may be overexpressed by introducing genetic constructs that code for the transcription factor thereby increasing the level of the transcription factor in transformed cells. Over-expression of YB-1 in a population of cells will tend to reduce apoptosis in the cells and can therefore be used to confer protection against agents that induce apoptosis, such as cytotoxic drugs. Genetic constructs for use in such methods comprise a polynucleotide encoding the transcription factor, together with suitable promoter and terminator sequences. Preferred polynucleotides for use in such genetic constructs include polynucleotides encoding a member of the Y-box family of binding proteins. Methods for making, introducing and expressing such genetic constructs, together with promoter and terminator sequences for use in such constructs are well known in the art.

As used herein, the phrase “contacting a population of cells with a genetic construct, anti-sense oligonucleotide or decoy oligonucleotide” includes any means of introducing a DNA molecule into any portion of one or more cells by any method compatible with cell viability and known to those of ordinary skill in the art. The cell or cells may be contacted in vivo, ex vivo, in vitro, or any combination thereof.

For in vivo uses, a genetic construct, anti-sense oligonucleotide or decoy oligonucleotide may be administered by various art-recognized procedures. See, e.g., Rolland, Crit. Rev. Therap. Drug Carrier Systems (1998) 15:143-198, and cited references. Both viral and non-viral delivery methods have been used for gene therapy. Useful viral vectors include adenovirus, adeno-associated virus (AAV), retrovirus, vaccinia virus and avian poxvirus. Improvements have been made in the efficiency of targeting genes to tumor cells with adenoviral vectors, for example, by coupling adenovirus to DNA-polylysine complexes and by strategies that exploit receptor-mediated endocytosis for selective targeting. See, e.g., Curiel et al., Hum. Gene Ther. (1992), 3:147-154; and Cristiano and Curiel, Cancer Gene Ther. (1996)-3:49-57. Non-viral methods for delivering polynucleotides are reviewed in Chang & Seymour, (Eds) Curr. Opin. Mol. Ther. (2000), vol. 2. These methods include contacting cells with naked DNA, cationic liposomes, or polyplexes of polynucleotides with cationic polymers and dendrimers for systemic administration (Chang & Seymour, Ibid.). Liposomes can be modified by incorporation of ligands that recognize cell-surface receptors and allow targeting to specific receptors for uptake by receptor-mediated endocytosis. See, for example, Xu et al., Mol. Genet. Metab. (1998), 64:193-197; and Xu et al., Hum. Gene Ther. (1999), 10:2941-2952.

Tumor-targeting bacteria, such as Salmonella, are potentially useful for delivering genes to tumors following systemic administration (Low et al., Nat. Biotechnol. (1999) 17:37-41). Bacteria can be engineered ex vivo to penetrate and to deliver DNA with high efficiency into mammalian epithelial cells in vivo and in vitro. See, e.g., Grillot-Courvalin et al., Nat. Biotechnol. (1998) 16:862-866. Degradation-stabilized oligonucleotides containing, for example, decoy oligonucleotides comprising YB-1 binding sites, may be encapsulated into liposomes and delivered to patients by injection either intravenously or directly into a target site. Alternatively, retroviral or adenoviral vectors, or naked DNA expressing anti-sense RNA for YB-1, may be delivered into patient's cells in vitro or directly into patients in vivo by appropriate routes. Suitable techniques for use in such methods are well known in the art.

The transcriptional regulators of apoptosis described herein have numerous uses and applications in addition to the therapeutical methods described above. Transcription factors that bind to regulatory regions of genes involved in apoptotic cell death are useful, for example, for studying regulation of transcription of such genes, and for modulating transcription and expression of coding portions of such genes, both in vitro and in vivo. CD95 transcription factors, such as YB-1, are useful for studying regulation of CD95 both in vitro and in vivo. For example, CD95 transcription factors are useful for identifying cell types and populations having CD95 transcription enhancing and/or silencing regulatory capabilities. Numerous techniques may be employed. Using CD95 regulatory polynucleotides as probes, for example, nuclear extracts from various cell sources may be screened by electrophoretic mobility shift assay (EMSA) for the presence or absence of the respective DNA/polypeptide complexes. Expression of transcription factors capable of binding to such probes can be directly assayed by amplifying a portion of their cDNAs, for example by polymerase chain reaction (“PCR”), or by detecting mRNA for these factors using DNA/RNA or RNA/RNA hybridization techniques, such as Northern analysis or RNase protection assays.

The word “polynucleotide(s),” as used herein, means a polymeric collection of nucleotides and includes DNA and RNA molecules, both sense and anti-sense strands, and comprehends cDNA, genomic DNA, and wholly or partially synthesized polynucleotides. It will be recognized that operable anti-sense polynucleotides may comprise a fragment of the full-length sequence, and the definition of “polynucleotide” therefore includes all such operable anti-sense fragments. Identification of human genomic DNA and heterologous species DNAs can be accomplished by standard DNA/DNA hybridization techniques, under appropriately stringent conditions, using all or part of a cDNA sequence as a probe to screen an appropriate library. Alternatively, PCR techniques using oligonucleotide primers that are designed based on known genomic DNA, cDNA and protein sequences can be used to amplify and identify genomic and cDNA sequences. Synthetic DNAs corresponding to the identified sequences and variants may be produced by conventional synthesis methods. All of the polynucleotides described herein are isolated and purified.

The word “polypeptide,” as used herein, encompasses amino acid chains of any length, including full length proteins, wherein amino acid residues are linked by covalent peptide bonds. Polypeptides of the present invention may be purified natural products, or may be produced partially or wholly using recombinant techniques. Such polypeptides may be glycosylated with mammalian or other eukaryotic carbohydrates or may be non-glycosylated. Polypeptides described herein may be produced recombinantly by inserting a DNA sequence that encodes the polypeptide into an expression vector and expressing the polypeptide in an appropriate host. Any of a variety of expression vectors known to those of ordinary skill in the art may be employed. Expression may be achieved in any appropriate host cell that has been transformed or transfected with an expression vector containing a polynucleotide that encodes a recombinant polypeptide. Suitable host cells include prokaryotes, yeast, and higher eukaryotic cells. Preferably, the host cells employed are E. coli, insect, yeast, or a mammalian cell line such as COS or CHO. The DNA sequences expressed in this manner may encode naturally occurring polypeptides, portions of naturally occurring polypeptides, or other variants thereof.

In general, the polypeptides disclosed herein are prepared in an isolated, substantially pure, form. Preferably, the polypeptides are at least about 80% pure, more preferably at least about 90% pure, and most preferably at least about 99% pure.

As used herein, the term “variant” comprehends nucleotide or amino acid sequences different from the specifically identified sequences, wherein one or more nucleotides or amino acid residues is deleted, substituted, or added. Variants may be naturally occurring allelic variants, or non-naturally occurring variants. In certain preferred embodiments, variants of the inventive sequences retain certain, or all, of the functional characteristics of the inventive sequence. Variant sequences (polynucleotide or polypeptide) preferably exhibit at least 50%, more preferably at least 75%, and most preferably at least 90% or 95% identity to a sequence of the present invention. The percentage identity is determined by aligning the two sequences to be compared as described below, determining the number of identical residues in the aligned portion, dividing that number by the total number of residues in the inventive (queried) sequence, and multiplying the result by 100.

Polynucleotide or polypeptide sequences may be aligned, and percentages of identical nucleotides in a specified region may be determined against another polynucleotide or polypeptide, using computer algorithms that are publicly available. Two exemplary algorithms for aligning and identifying the similarity of polynucleotide sequences are the BLASTN and FASTA algorithms. The alignment and similarity of polypeptide sequences may be examined using the BLASTP algorithm. BLASTX and FASTX algorithms compare nucleotide query sequences translated in all reading frames against polypeptide sequences. The BLASTN, BLASTP and BLASTX algorithms are available on the NCBI anonymous FTP server and are available from the National Center for Biotechnology Information (NCBI), National Library of Medicine, Building 38A, Room 8N805, Bethesda, Md. 20894 USA.

The FASTA and FASTX algorithms are available on the Internet. The FASTA software package is also available from the University of Virginia by contacting the Assistant Provost for Research, University of Virginia, PO Box 9025, Charlottesville, Va. 22906-9025. The FASTA algorithm, set to the default parameters described in the documentation and distributed with the algorithm, may be used in the determination of polynucleotide variants. The readme files for FASTA and FASTX v1.0x that are distributed with the algorithms describe the use of the algorithms and describe the default parameters. The use of the FASTA and FASTX algorithms is also described in Pearson, and Lipman, Proc. Natl. Acad. Sci. USA 85:2444-2448, 1988; and Pearson, Methods in Enzymol. 183:63-98, 1990.

The BLASTN algorithm version 2.0.4 [Feb. 24, 1998], 2.0.6 [Sep. 16, 1998] and 2.0.11 [Jan. 20, 2000], set to the default parameters described in the documentation and distributed with the algorithm, is preferred for use in the determination of polynucleotide variants according to the present invention. The BLASTP algorithm version 2.0.4, 2.0.6 and 2.0.11, set to the default parameters described in the documentation and distributed with the algorithm, is preferred for use in the determination of polypeptide variants according to the present invention. The use of the BLAST family of algorithms, including BLASTN, BLASTP and BLASTX is described in the publication of Altschul, et al., Nucleic Acids Res. 25:3389-3402, 1997.

The following running parameters are preferred for determination of alignments and similarities using BLASTN that contribute to the E values and percentage identity for polynucleotides: Unix running command with default parameters thus: blastall -p blastn -d embldb -e 10 -G 0 -E 0 -r 1 -v 30 -b 30 -i queryseq -o results; and parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -r Reward for a nucleotide match (blastn only) [Integer]; -v Number of one-line descriptions (V) [Integer]; -b Number of alignments to show (B) [Integer]; -i Query File [File In]; -o BLAST report Output File [File Out] Optional. The following running parameters are preferred for determination of alignments and similarities using BLASTP that contribute to the E values and percentage identity for polypeptides: blastall -p blastp -d swissprotdb -e 10 -G 1 -E 11 -r 1 -v 30 -b 30 -i queryseq -o results; and the parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -v Number of one-line descriptions (v) [Integer]; -b Number of alignments to show (b) [Integer]; -I Query File [File In]; -o BLAST report Output File [File Out] Optional.

The “hits” to one or more database sequences by a queried sequence produced by BLASTN, BLASTP, FASTA, or a similar algorithm, align and identify similar portions of sequences. The hits are arranged in order of the degree of similarity and the length of sequence overlap. Hits to a database sequence generally represent an overlap over only a fraction of the sequence length of the queried sequence.

As noted above, the percentage identity of a polynucleotide or polypeptide sequence is determined by aligning polynucleotide and polypeptide sequences using appropriate algorithms, such as BLASTN or BLASTP, respectively, set to default parameters; identifying the number of identical nucleic or amino acids over the aligned portions; dividing the number of identical nucleic or amino acids by the total number of nucleic or amino acids of the polynucleotide or polypeptide of the present invention; and then multiplying by 100 to determine the percentage identity. By way of example, a queried polynucleotide having 220 nucleic acids has a hit to a polynucleotide sequence in the EMBL database having 520 nucleic acids over a stretch of 23 nucleotides in the alignment produced by the BLASTN algorithm using the default parameters. The 23 nucleotide hit includes 21 identical nucleotides, one gap and one different nucleotide. The percentage identity of the queried polynucleotide to the hit in the EMBL database is thus 21/220 times 100, or 9.5%. The identity of polypeptide sequences may be determined in a similar fashion.

The BLASTN and BLASTX algorithms also produce “Expect” values for polynucleotide and polypeptide alignments. The Expect value (E) indicates the number of hits one can “expect” to see over a certain number of contiguous sequences by chance when searching a database of a certain size. The Expect value is used as a significance threshold for determining whether the hit to a database indicates true similarity. For example, an E value of 0.1 assigned to a polynucleotide hit is interpreted as meaning that in a database of the size of the EMBL database, one might expect to see 0.1 matches over the aligned portion of the sequence with a similar score simply by chance. By this criterion, the aligned and matched portions of the sequences then have a probability of 90% of being the same. For sequences having an E value of 0.01 or less over aligned and matched portions, the probability of finding a match by chance in the EMBL database is 1% or less using the BLASTN algorithm. E values for polypeptide sequences may be determined in a similar fashion using various polypeptide databases, such as the SwissProt database.

According to one embodiment, “variant” polynucleotides and polypeptides, with reference to each of the polynucleotides and polypeptides disclosed herein, preferably comprise sequences having the same number or fewer nucleic or amino acids than each of the polynucleotides or polypeptides of the present invention and producing an E value of 0.01 or less when compared to the polynucleotide or polypeptide of the present invention. That is, a variant polynucleotide or polypeptide is any sequence that has at least a 99% probability of being the same as the polynucleotide or polypeptide of the present invention, measured as having an E value of 0.01 or less using the BLASTN or BLASTX algorithms set at the default parameters. According to a preferred embodiment, a variant polynucleotide is a sequence having the same number or fewer nucleic acids than a polynucleotide of the present invention that has at least a 99% probability of being the same as the polynucleotide of the present invention, measured as having an E value of 0.01 or less using the BLASTN algorithm set at the default parameters. Similarly, according to a preferred embodiment, a variant polypeptide is a sequence having the same number or fewer amino acids than a polypeptide of the present invention that has at least a 99% probability of being the same as the polypeptide of the present invention, measured as having an E value of 0.01 or less using the BLASTP algorithm set at the default parameters.

Variant polynucleotide sequences will generally hybridize to the recited polynucleotide sequences under stringent conditions. As used herein, “stringent conditions” refers to prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

The present invention also encompasses polynucleotides that differ from the disclosed sequences but that, as a consequence of the degeneracy of the genetic code, encode a polypeptide which is the same as that encoded by a polynucleotide disclosed herein. Additionally, polynucleotides comprising sequences that differ from the polynucleotide sequences disclosed herein as a result of deletions and/or insertions totaling less than 10% of the total sequence length are also contemplated by and encompassed within the present invention. Similarly, polypeptides comprising sequences that differ from the polypeptide sequences disclosed herein, as a result of amino acid substitutions, insertions, and/or deletions totaling less than 10% of the total sequence length are contemplated by and encompassed within the present invention.

Transcription factors are described herein with reference to activities involving “enhancing” or “silencing” transcription of coding portions of a gene appropriately linked to a regulatory polynucleotide sequence and a suitable promoter. Such regulatory activities are observed and may be assessed both in vitro and in vivo. It will be recognized that organisms and cells of different types, as well as cells in different developmental stages and physiological or in vitro conditions, may exhibit substantially different transcriptional activities. Transcriptional activity is considered to be “enhanced” or silenced” when there is at least a 50%, and more preferably at least a 100%, change in the level of transcriptional activity in the presence of a transcription factor or polynucleotide/transcription factor complex compared to the transcriptional activity measured under substantially the same conditions in the absence of the transcription factor or polynucleotide/transcription factor complex. Binding of polypeptide transcription factors to polynucleotides and formation of DNA/polypeptide complexes may be assessed in vitro using standard EMSA techniques described below, or in vivo by measuring enhancement or silencing of transcription from a gene appropriately linked to a regulatory polynucleotide and a suitable promoter.

The phrase “transcriptional regulator of apoptosis” is used herein to refer to a transcription factor that binds to (or interacts with) and regulates transcription and/or translation of a polynucleotide involved in apoptosis. The phrase “target polynucleotide” as used herein encompasses both DNA and RNA sequences. The phrase “transcriptional regulator of apoptosis target polynucleotide” is used herein to refer to a polynucleotide involved in apoptosis with which the transcriptional regulator of apoptosis binds or interacts. The phrase “DNA-damaging agent” is used herein to refer to any chemical or physical agent that is capable of inducing the formation of intra- or inter-strand crosslinks in DNA.

The present invention is illustrated by reference to the following experimental protocols and results. The experimental protocols and results support the specification and claims and should not be construed to limit the invention, as claimed, in any fashion.

### EXAMPLE 1

### Identification of Transcription Factors which Bind to Regulatory hCD95 Polynucleotides

The identification of regulatory human CD95 polynucleotides is described in U.S. Pat. No. 5,912,168, the disclosure of which is hereby incorporated by reference in its entirety. The transcription enhancer region denominated E1 (SEQ ID NO: 1) resides between nucleotide positions −1007 and −964 in the hCD95 gene, and the transcription silencer region denominated S1 (SEQ ID NO: 2) resides between nucleotide positions −1035 and −1008 in the hCD95 gene. These regions mediate cell type-specific and activation state-dependent transcriptional regulation of the CD95 gene during activation-induced cell death.

Electrophoretic Mobility Shift Assay (EMSA) Protocol

Nuclear extracts were prepared from Jurkat (human T lymphoma cells) and MP-1 (human EBV-transformed B cells) grown under 5% CO₂ in RPMI 1640 medium supplemented with antibiotics and 5% fetal bovine serum, and from HeLa, COS-7, CV-1 (COS-7 derivative) and L929 (murine fibroblast cells) grown under 10% CO₂ in DMEM medium supplemented with antibiotics and 5% fetal bovine serum according to the method of Andrews and Faller, Nucleic Acids Research, Vol. 19, No. 9, 1991. Unless otherwise indicated, binding reactions contained 5 μg nuclear extract (adjusted to give an equal contribution of 40 mM NaCl in the binding reaction), 150 mM (or 100 mM) KCl, 2 μg of non-specific competitor DNA (poly[d(1-C)] or poly[d(A-T)], as indicated), 12% glycerol, 12 mM Hepes pH 7.9, 4 mM Tris-HCl pH 7.9, 1 mM EDTA, 1 mM dithiothreitol, and 20 fmole of [γ-³²P]ATP-labeled probe (double- or single-stranded, as indicated). The indicated amounts of competitor oligonucleotides were added before addition of the nuclear extract and the reaction incubated for 30 min. at room temperature. Three μl loading buffer (12% glycerol, 12 mM Hepes pH 7.9, 4 mM Tris-HCI pH 7.9, 1 mM EDTA, 1 mM dithiothreitol, 0.1% bromophenol blue) were added, and the reactions loaded on pre-run (2 h at 150 V) non-denaturing 4% polyacrylamide gels (acrylamide:bisacrylamide, 30:1). The gels were run in 50 mM Tris-HCI (pH 8.5), 380 mM glycine, 2 mM EDTA at 150 V (constant voltage) with water-cooling. Gels were dried and autoradiographed for 1 to 4 days. This assay, and variants of this assay are referred to herein as the “standard EMSA assay protocol.”

Identification of Transcription Factors which Bind to hCD95 Enhancer Region

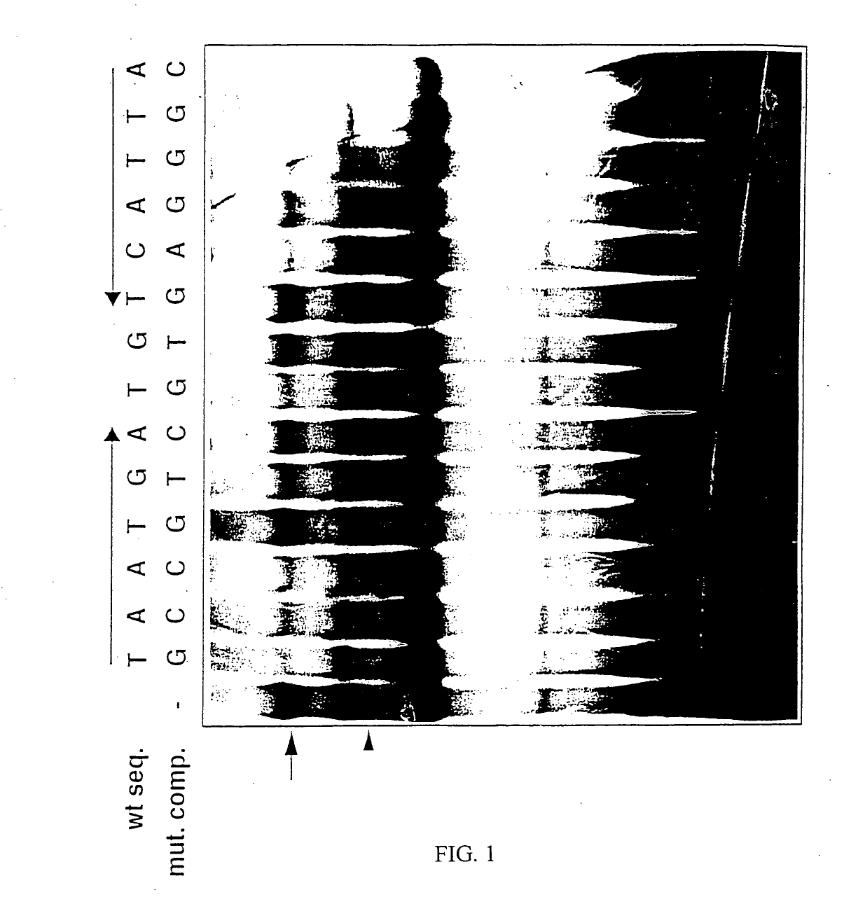

EMSA analysis using an E1 double-stranded probe (referred to as E1 probe; SEQ ID NO: 8) and Jurkat cell nuclear extract revealed that a hexameric inverted repeat nucleotide contained within E1 (referred to as IR2; SEQ ID NO: 5) mediates sequence-specific binding of nuclear factors. Experimental results are shown in FIG. 1. Distinct DNA/polypeptide complexes formed with these nuclear factors, referred to herein as transcription factors, are marked by an arrow and an arrowhead. Transcription factors that bind to the enhancer region hexameric inverted repeat of SEQ ID NO: 5 were also present in murine L929 cells and other primate and rodent cells, including HeLa, MP-1, COS-7, and rat dermal papilla (rDP) cells.

Mutational scanning of the enhancer region hexameric inverted repeat of SEQ ID NO: 5, using a 50-fold molar excess of double stranded competitor oligonucleotides containing the single nucleotide substitutions indicated above the respective lanes (derivatives of SEQ ID NO: 8) in EMSA analysis together with the wildtype enhancer probe (SEQ ID NO: 1, See FIG. 1) demonstrated the importance of individual nucleotides for binding and identified the degenerate E1 consensus motif identified in SEQ ID NO: 3 as an hCD95 enhancer region (E1) binding site.

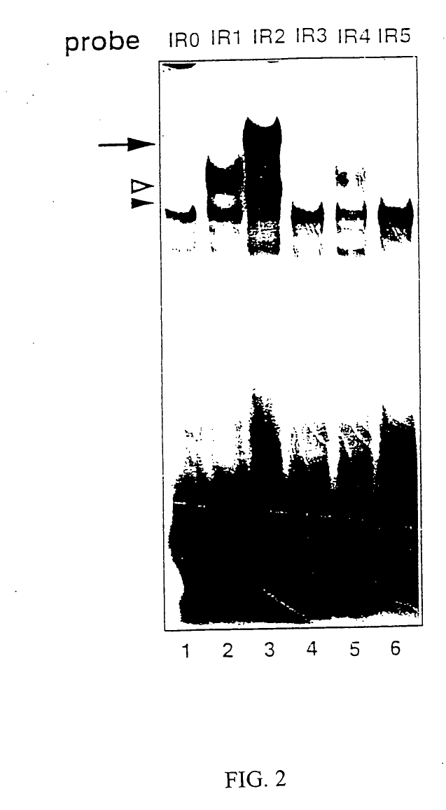

Using nuclear extracts from murine L929 cells in EMSA analysis, sequence-specific formation of novel DNA/polypeptide complexes which were different from enhancer region binding site IR2 complexes, was demonstrated with enhancer region sequence motifs spaced by 1 bp (referred to as IR1; SEQ ID NO: 4) and 4 bp (referred to as IR4; SEQ ID NO: 6). Experimental results are shown in FIG. 2. Complexes formed by the nuclear transcription factors that bind to the hexameric inverted repeat-containing enhancer probe (SEQ ID NO: 8), are marked by an arrow and arrowhead. The open arrowhead indicates the presence of complexes formed by the enhancer region spacing derivatives (IR1; SEQ ID NO: 4 and IR4; SEQ ID NO: 6). The enhancer region elements IR1, IR2 and IR4 cross-competed for the formation of the respective DNA/polypeptide complexes in a sequence-specific manner. These results indicate the existence of a family of related transcription factors which recognize the same CD95 enhancer region binding motif but have different spacing requirements.

Concatamerized enhancer (SEQ ID NO: 8) sequences were used as probes to screen cDNA expression libraries according to methods published in Singh et al., (1989) BioTechniques, 7:252-261. Using a single-stranded 3× E1 (3× SEQ ID NO: 8) probe to screen a HeLa expression library, clones encoding the following DNA-binding proteins were isolated: human YB-1 (EMBL Accession No. M24070; SWISS-PROT Accession No. P16990); and human hnRNP D (EMBL Accession No. D55672; SWISS-PROT Accession Nos. Q14101 and Q14103).

Identification of Transcription Factors which Bind to the hCD95 Silencer Region

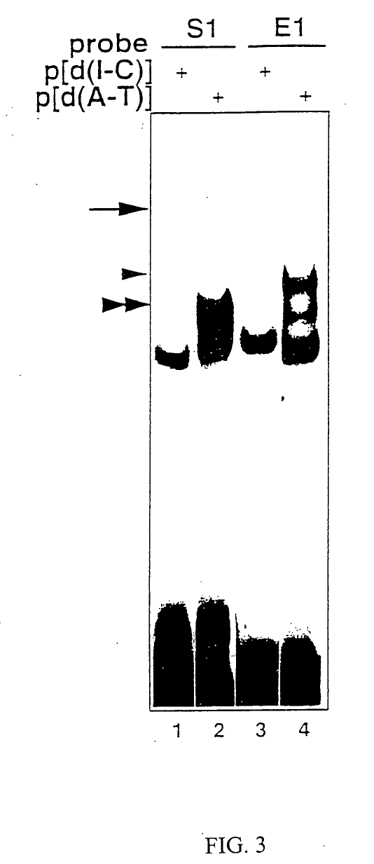

Novel DNA/polypeptide complexes were formed using a silencer region probe (S1; SEQ ID NO: 2), and an enhancer region IR2 probe (SEQ ID NO: 8) when using polydAdT instead of polydIdC as non-specific competitor DNA in EMSA analysis. Results are shown in FIG. 3, with the silencer probe identification shown above the lanes and the double arrowhead indicating the novel complex. This factor(s) also bound to single-stranded silencer probes or was competed out by single-stranded silencer and enhancer probes.

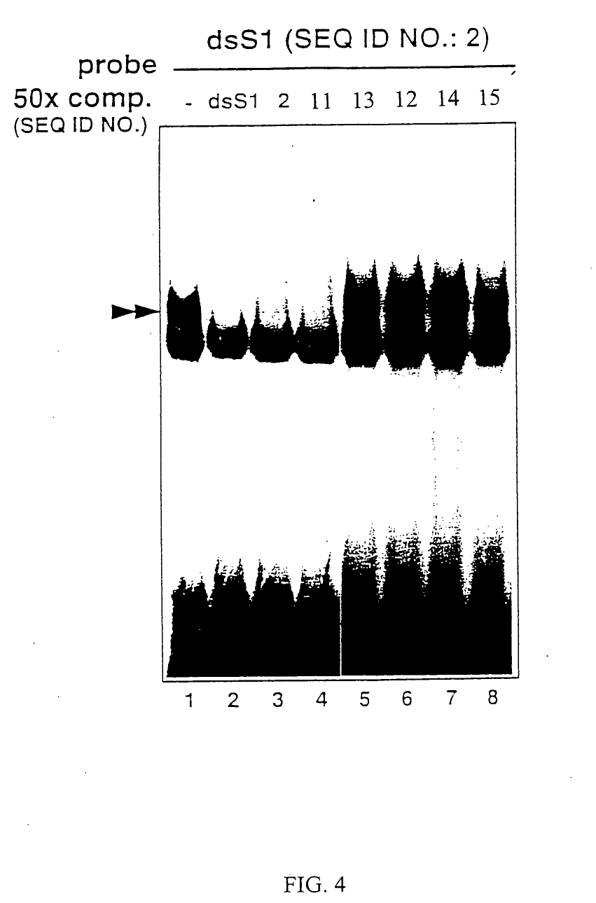

Further EMSA analysis is illustrated in FIG. 4, using silencer probes having SEQ ID NO: 2 and 11-15. The silencer region heptamer motif (SEQ ID NO: 7 or 17), which is present in identical copies in the S1 and E1 regions, appeared to mediate interaction with the transcription factor(s), since the respective DNA/polypeptide complex was also formed with a single-stranded silencer region probe (SEQ ID NO: 9) including the silencer region heptamer sequence, but not with the complement of this probe (SEQ ID NO: 10). Silencer region probes from both DNA strands, having an interruption of the heptamer motif in the silencer region and containing less than a full heptamer motif (identified in SEQ ID NO: 12-15) showed greatly reduced ability to compete for complex formation with the wildtype S1 probe.

Concatamerized silencer (SEQ ID NO: 2) sequences were used as probes to screen cDNA expression libraries as described above. Using a single-stranded 3× S1 (3× SEQ ID NO: 2) probe to screen HeLa and rat dermal papilla expression libraries, clones encoding DNA-binding proteins were isolated. The isolated clones included human YB-1 (EMBL Accession No. M24070; SWISS-PROT Accession No. P16990) and rat YB-1 (EMBL Accession No. M57299; SWISS-PROT Accession No. P22568).

To confirm that YB-1 is a component of the CD95 silencer complex, a YB-1 antibody was incubated with nuclear extracts and the silencer regulatory sequence (S1; SEQ ID NO: 2) probe using supershift procedures documented in Macdonald et al. (1995) J. Biol. Chem., 270: 3527-3533. Incubation with the antibody caused a supershift, indicating that YB-1 is a component of the silencer complex.

Characterization of Transcription Factors for Silencer/Enhancer Regions by UV-Crosslinking

UV-crosslinking was performed essentially as described by Miyamoto et al., Methods Enzymol. 254:633-641, 1995. Oligonucleotides of 44 and 28 bases in length were end-labeled as in the above-described EMSA reactions. Double-stranded DNA probes were prepared by annealing the end-labeled oligonucleotides and filling in with [γ-³²P]dATP, [γ-³²P]dCTP, [γ-³²P]dGTP (800 Ci/mmol) and 5-bromo-2′-dUTP using Klenow (Miyamoto et al., 1995.)

A standard EMSA binding reaction was set up with 40 fmol probe and 10 μg nuclear extracts ±4 pmol competitor DNA in a total volume of 40 ml in a flat-bottomed microtitre plate. The plate was covered with Saran-wrap and placed on ice. The reactions were irradiated for 60 minutes by inverting a UV transilluminator of 305 nm wavelength, such that the illuminator was within 5 cm from the microtitre plate. The reactions were then divided into two. One aliquot was run on a 4% non-denaturing gel as described previously, and the second aliquot was run on a 10% reducing SDS-PAGE gel with ¹⁴C-labeled protein markers. The gels were dried followed by autoradiography with an intensifying screen for 1-3 days.

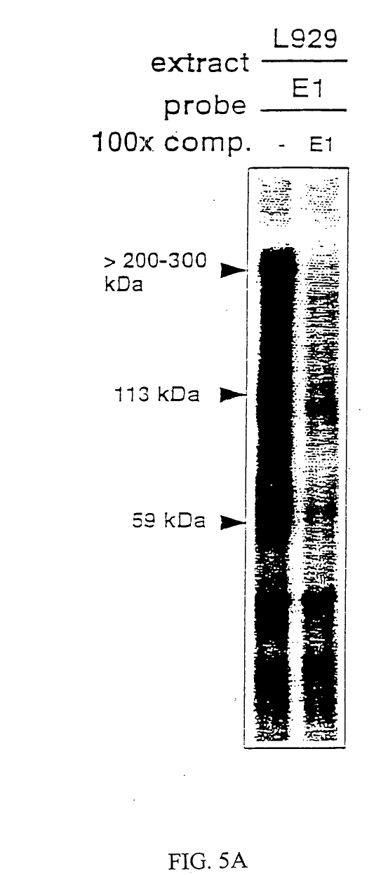

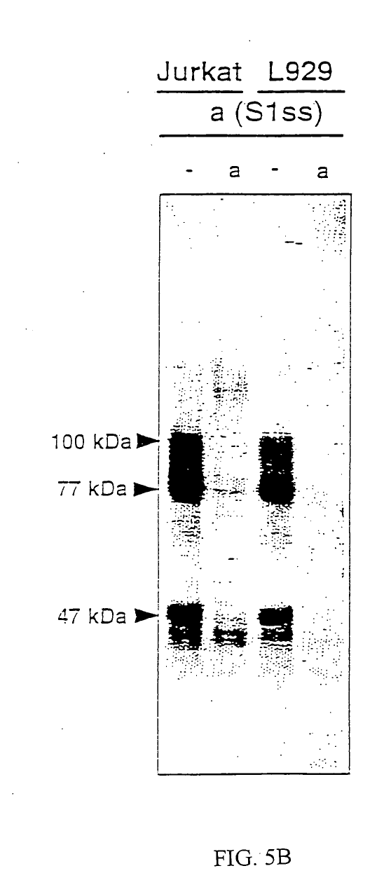

As shown in FIG. 5B, UV-crosslinking analysis using an end-labeled, single-stranded S1 probe (SEQ ID NO: 2), revealed cross-linked DNA/polypeptide complexes of approximately 47, 77 and 100 kDa in Jurkat and L929 cells. Results from probing a Southwestern blot of Jurkat cell nuclear extract with the single-stranded S1 probe (SEQ ID NO: 2) suggested that the 47 kDa and 100 kDa complexes corresponded to single nuclear proteins. UV-crosslinking with a double-stranded E1 probe (SEQ ID NO: 1), shown in FIG. 5A, revealed cross-linked DNA/polypeptide complexes of approximately 59 kDa, 113 kDa, and a high molecular weight complex of approximately 200 to 300 kDa in L929 cells.

Characterization of Transcription Factors for Silencer/Enhancer Regions by Southwestern Analysis

20-40 μg of nuclear extracts from Jurkat, L929 and rat dermal papilla (rDP) cells, prepared as described above, were electrophoresed on a 8-10% reducing SDS-PAGE gel with ¹⁴C-labeled protein markers. The gel was pre-soaked in Transfer Buffer prior to electroblotting to 0.2 mm nitrocellulose filters as described by Li and Desiderio, Appendix 1, “Transcription Factors: A Practical Approach” (D. S. Latchman, Ed.) IRL Press, Oxford, pp. 187-196, 1993. Nitrocellulose filters were blocked in 2.5% (w/v) dried milk powder, 25 mM Hepes (pH 8), 1 mM DTT, 10% (v/v) glycerol, 50 mM NaCl, 1 mM EDTA at 4° C. for 18 hours. Filters were hybridized in SW-Binding Buffer (12% (v/v) glycerol, 12 mM Hepes (pH 8), 4 mM Tris-HCI (pH 8), 1 mM EDTA, 1 mM DTT, 40 mM NaCl, 100 mM KCl), 1 pmol/ml ³²P-labeled DNA probe (end labeled or filled in, as above), 10 μg/ml non-specific competitor DNA (poly[d1-dC] or poly[dA-dT]) and ±100 pmol/ml competitor DNA for 60 minutes at room temperature. The filters were washed for 4×7 minutes in SW-Binding Buffer at 4° C., prior to autoradiography for 3 days with an intensifying screen.

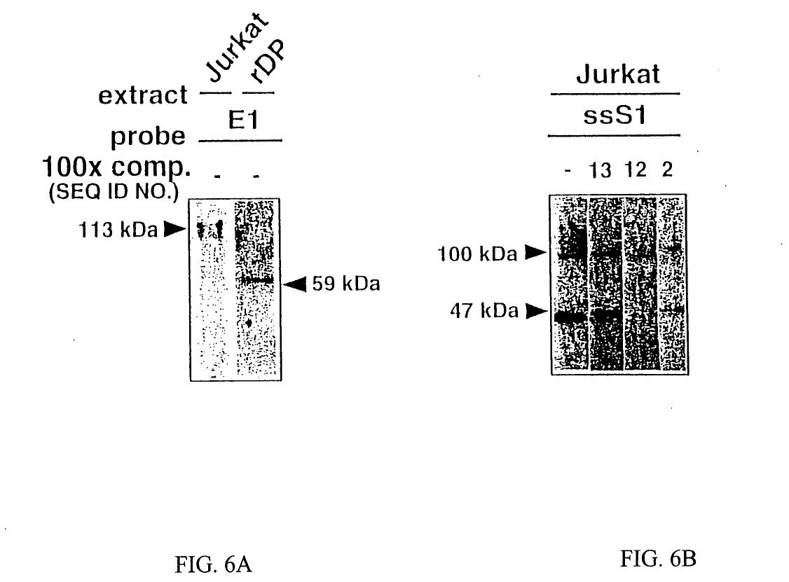

Southwestern analysis results of Jurkat and rDP nuclear extracts using a double stranded enhancer probe (SEQ ID NO: 8) are illustrated in FIG. 6A. These results show protein species having molecular weights of approximately 59 kDa (rDP) and 113 kDa (Jurkat and rDP) to which a double-stranded enhancer probe binds. The results of Southwestern analysis of Jurkat nuclear extracts using a single stranded silencer probe (SEQ ID NO: 2) are illustrated in FIG. 6B. These results show protein species having molecular weights of approximately 47 kDa and 100 kDa to which a single-stranded silencer probe binds. Binding of the silencer probe to these proteins is greatly reduced or absent in the presence of a heptamer (SEQ ID NO: 7)-containing competitor (SEQ ID NO: 9), complementary to the probe strand, but not an equivalent competitor (SEQ ID NO: 10) corresponding to the probe strand. These Southwestern results are consistent with UV-crosslinking and EMSA results obtained with the probes and competitors described above.

Functional Analysis of Transcription Factors

The CD95 silencer (SEQ ID NO: 2), enhancer (SEQ ID NO: 8) and promoter fragment (SEQ ID NO: 16) were cloned in front of the HSV tk promoter and CAT gene in the reporter plasmid pBLCAT8⁺. Constructs encoding the YB-1 protein were cloned into the vector pCDNA3. The constructs were overexpressed in HeLa cells with both of the CD95 promoter-CAT reporter constructs. The overexpression of sense YB-1 repressed transcription of the CD95 promoter 7-fold and expression of anti-sense YB-1 stimulated transcription of the CD-95 promoter 2-fold. This analysis suggests that YB-1 plays a role in repression of the CD95 promoter in vivo.

EMSA Analysis of Silencer/DNA and Enhancer/DNA Complexes

EMSA gel mobility shift assays using the protocol described above determined several characteristics of the silencer/DNA and enhancer/DNA complexes. The half-life of both silencer/DNA and enhancer/DNA complexes is approximately one hour. The enhancer/DNA complex was only stable in less than 300 mM KCl, and formation of the enhancer/DNA complex required the presence of divalent cations. Thirty percent (30%) of the silencer/DNA complex was stable in 2M KCl. The formation of the silencer/DNA complex did not require the presence of divalent cations, and the silencer/DNA complex contained an ATP-dependent protein.

These experimental results demonstrate that the silencer/DNA complex is very stable, and the enhancer/DNA complex is much less stable. This is consistent with the anticipated in vivo activity, i.e. the silencer/DNA complex is formed most of the time, and expression of CD95 is suppressed. The ATP-dependence suggests that ATP is utilized to unwind the CD95 silencer regulatory region to provide the single stranded conformation to which the transcription factors bind.

### EXAMPLE 2

### Modulation of Apoptotic Cell Death In Vitro by YB-1 or PURα

In Vitro Assays Using Hep G2 Cells

Modulation of apoptotic cell death by regulating the binding of YB-1 or Pur α to the CD95 promoter was demonstrated in the liver carcinoma cell line Hep G2 using two different techniques, namely anti-sense oligonucleotides and decoy oligonucleotides.

Hep G2 cells were transfected with pools containing five anti-sense phosphorothioate oligos, of 22 nucleotides each, to either YB-1 or Pur α. Cells were transfected with 100 μM of oligonucleotides (pools 1, 2, 3, 4 and a non-specific oligo) using the published techniques of Stewart et al., Biochem Pharmacol., 51:461-469, 1996. The rate of cell death was determined using MTT as a substrate according to the methods of Vistica et al., Cancer Res. 48:4827, 1991.

Pool 1 consisted of five 22-mer oligonucleotides to the anti-sense strand of YB-1 identified as SEQ ID NO: 29-33. Pool 2 consisted of five 22-mer oligonucleotides to the anti-sense strand of YB-1 identified as SEQ ID NO: 19-23. Transfection of Hep G2 cell cultures with pools 1 and 2 resulted in Hep G2 cell death of 69% and 75%, respectively, 48 hours following transfection. Pool 3 consisted of five 22-mer oligonucleotides to the anti-sense strand of Pur α, identified as SEQ ID NO: 34-38. Pool 4 consisted of five 22-mer oligonucleotides to the anti-sense strand of Pur α, identified as SEQ ID NO: 24-28. Transfection of Hep G2 cell cultures with pools 3 and 4 resulted in cell death of 66% and 70%, respectively. Cell death rates of 0% and 8% were recorded 48 hours following transfection in control samples containing no oligos or a nonspecific phosphorothioate oligo, respectively. By inhibiting translation of YB-1 and Pur α, the amount of endogenous YB-1 or Pur α available to bind to the CD95 silencer regulating region was reduced, CD95 expression was up-regulated, and increased apoptotic cell death resulted.

In another experiment, Hep G2 cells were transfected with phosphorothioate oligos containing the silencing regulatory regions identified in SEQ ID NO: 2 and 11 using the techniques described above. Cell death rates of 59% and 63% were recorded in samples transfected with phosphorothioate oligos containing the silencing regulatory regions identified in SEQ ID NO: 2 and 11, respectively, with cell death rates of 0% and 8% being recorded in control samples containing no oligos or a nonspecific phosphorothioate oligo, respectively. By introducing a phosphorothioate oligo that competed with endogenous CD95 regulatory sequences (also known as a decoy oligo), the amount of transcription factors (such as YB-1) available to bind to the endogenous CD95 silencer regulatory regions was reduced, CD95 expression was up-regulated, and increased apoptotic cell death resulted.

In Vitro Assays Using Hep G2, A549, SK-Mel 5, and Non-Malignant Cells

Phosphorothioate oligonucleotides were designed to the anti-sense strand of YB-1 (pool 2) and the anti-sense strand of Pur α (pool 4). Pool 2 consisted of five 22-mer oligonucleotides identified as SEQ ID NO: 19-23. Pool 4 consisted of five 22-mer oligonucleotides identified as SEQ ID NO: 24-28. Oligos constructed to the upper or lower strand of the CD95 silencer region, identified as oligos “57” and “58,” were both 28-mers and corresponded to SEQ ID NO: 2 and 11, respectively. Finally, a non-specific 22-mer oligonucleotide, designated “oligo 81”, was designed to a non-specific sequence of DNA.

Cell cultures of non-malignant neonatal foreskin fibroblasts were transfected with 100 μM of each oligonucleotide (pool 2, pool 4, and oligos 57, 58 and 81) using the published techniques of Stewart et al., Biochem Pharmacol., 51:461-169, 1996. The rate of cell death was determined using MTT as a substrate according to the methods of Vistica et al., Cancer Res., 48:4827, 1991. No cell death was detected following transfection of the non-malignant neonatal foreskin fibroblasts.

Three malignant cell lines were tested in a similar fashion: Hep G2 (human liver carcinoma cell line), A549 (human lung carcinoma cell line) and SK-Mel 5 (human melanoma cell line). Cultures of each cell line were transfected with 100 μM of each oligonucleotide (pool 2, pool 4, oligos 57, 58 and 81) and the rate of cell death was determined as described above. Cell death rates at 48 or 72 hours following transfection are shown in Table I below. As shown below, transfection of the malignant cell lines with pools 2 and 4, and oligos 57 and 58, resulted in significant rates of cell death.

TABLE I

| | | % Cell | % Cell | |
|---|---|---|---|---|
| | | Death | Death | % Cell Death |
| Oligo | Oligo/s | Hep G2 | A549 | SK-Mel 5 |
| Description | Transfected | (48 hours) | (72 hours) | (72 hours) |
| anti-sense YB-1 | pool 2 | 75 | 44 | 42 |
| anti-sense Pur α | pool 4 | 89 | 63 | 44 |
| SEQ ID NO: 2 | 57 | 59 | 65 | 74 |
| SEQ ID NO: 11 | 58 | 65 | 66 | 14 |
| non-specific | 81 | 8 | 0 | 12 |
| | no DNA | 0 | 0 | 0 |

In order to confirm that YB-1 levels were being modulated, biotinylated forms of the decoy (SEQ ID NO: 11) and negative control oligonucleotides (oligo 81) were used to transfect SK-Mel-5 cells. Cell lysates were incubated with streptavidin beads to isolate the oligonucleotide/protein complexes formed in the cell. The remaining cell supernatants were analyzed by Western blotting with an antibody to YB-1. The results confirmed that the levels of free YB-1 were reduced in cells transfected with the decoy oligonucleotide (SEQ ID NO: 11) compared to control oligonucleotide transfectants. To demonstrate that the anti-sense YB-1 oligonucleotides (SEQ ID NO: 19-23) reduced levels of YB-1 protein, cell lysates from these and negative control oligonucleotide (oligo 81) transfectants were analyzed with anti-YB-1 antibody. YB-1 levels were found to be reduced in cells transfected with the anti-sense YB-1 oligonucleotides.

By inhibiting translation of YB-1 and Pur α using the anti-sense oligos in pools 2 (SEQ ID NO: 19-23) and 4 (SEQ ID NO: 24-28), the amount of endogenous YB-1 or Pur α available to bind to polynucleotides involved in apoptosis was reduced, and increased apoptotic cell death resulted. Transfection with oligos 57 and 58, corresponding to the CD95 silencer regulatory sites identified in SEQ ID NO: 2 and 11, led to increased apoptotic cell death.

These experiments demonstrate that malignant cells, but not non-malignant cells, are killed by transfection with either anti-sense oligonucleotides to YB-1 and Pur α or with decoy oligonucleotides.

### EXAMPLE 3

### Modulation of Apoptic Cell Death In Vivo by YB-1

The ability of YB-1 to modulate apoptotic cell death in vivo was examined as follows.

Mouse fibrosarcoma cells were co-injected with the following oligonucleotides: (1) negative control oligonucleotide (oligo 81); (2) two decoy oligonucleotides corresponding to the 5′ to 3′ strand and 3′ to 5′ strand, respectively, of the CD95 silencer regulatory sites identified in SEQ ID NO: 2 and 11; and (3) a pool consisting of the YB-1 anti-sense oligonucleotides provided in SEQ ID NO: 20 and 21. Groups of three mice were injected with either the cells plus oligonucleotides or with cells alone under the skin and the mice were observed for five weeks.

All the mice treated with either fibrosarcoma cells plus negative control oligonucleotide or cells only grew tumors about 1.5 cm in diameter. No tumors were apparent in any of the mice treated with either the decoy or anti-sense oligonucleotides. These results demonstrate that reducing the amount of available YB-1 is effective in increasing apoptotic cell death and thereby reducing the growth of tumors.

### EXAMPLE 4

### Depletion of YB-1 Induces Apoptosis by Activating p53

It is known that SK-Mel-5 cells express high levels of CD95 ligand. In order to examine whether YB-1 induced apoptosis was due to CD95/CD95L interactions, SK-Mel-5 cells tranfected with either anti-sense oligonucleotides to YB-1 or YB-1 decoy oligonucleotides were allowed to recover in the presence of soluble CD95:Fc or a neutralizing CD95 antibody. Both these reagents have been shown to impede CD95-mediated apoptosis. The results demonstrated that apoptosis was not abrogated with either CD95:Fc or the neutralizing antibody, indicating that apoptosis was not induced via the CD95/CD95L pathway. To determine whether other cultured tumor cells were susceptible to apoptosis induced by the depletion of YB-1, a panel of tumor cell lines plus two normal cell lines were transfected with anti-sense oligonucleotides to YB-1, decoy oligonucleotides, or negative control oligonucleotides as described above. These results are summarized in Table II, below.

TABLE II

| | | | Induction of |
|---|---|---|---|
| Cell Line | Source | p53 Status | Apoptosis |
| SK-Mel-5 | Melanoma | Wild-type | Yes |
| HepG2 | Hepatocarcinoma | Wild-type | Yes |
| A549 | Adenocarcinoma | Wild-type | Yes |
| NZM9 | Melanoma | Wild-type | Yes |
| RKO | Colon carcinoma | Wild-type | Yes |
| B10.2 | Fibrosarcoma | Wild-type | Yes |
| Jurkat | T-cell leukemia | Mutant | No |
| U937 | Histiocytic lymphoma | Null | No |
| HL60 | Promyelocytic | Null | No |
| | leukemia | | |
| RKO p53.13 | Colon carcinoma | Dom. Neg. | No |
| HaCaT | Transformed | Mutant | No |
| | keratinocyte | | |
| Fibroblasts | Neonatal foreskin | Wild-type | No |
| Melanocytes | Neonatal foreskin | Wild-type | No |

These results indicate that a wide range of cells lines derived from many tumor types were susceptible to apoptosis induced by YB-1 depletion. However, a number of cell lines were not susceptible to apoptosis. These cell lines have either mutant p53 or are lacking in p53, and these results therefore indicated a correlation between p53 status and apoptotic susceptibility. In order to directly test the involvement of p53, two derivatives of the human colon carcinoma cell line RKO were transfected with the decoy and negative control oligonucleotides. The original RKO cell line has wild type p53, but a modified line, termed RKO p53.13, overexpressed a murine dominant negative p53. The RKO cell line (wild type p53) was susceptible to apoptosis induced by YB-1 depletion, with 53% cell death compared to the control oligonucleotide transfectants. However, the presence of the dominant negative form of p53 (RKO p53.13) protected these cells from death, with no additional apoptosis being observed in cells depleted of YB-1.

To support a direct involvement of p53 in the apoptosis pathway, transfected SK-Mel-5 cells were depleted of both YB-1 and p53. These cells were transfected with the decoy oligonucleotide, in the absence or the presence of a double-stranded p53 cis element from the human gadd45 promoter. Depletion of both YB-1 and p53 abrogated apoptosis, whereas a double-stranded negative control co-transfected with the decoy oligonucleotide had no effect. These results directly associate p53 with the apoptotic pathway induced by YB-1 depletion.

To determine whether YB-1 transcriptionally regulates p53 gene expression, reporter gene assays were performed. Murine p53 promoter fragments linked to a CAT reporter gene were co-transfected with YB-1 into SK-Mel-5 cells. YB-1 was able to repress expression from the p53 promoter construct to background levels. Levels of expression from an E3 adenovirus promoter construct were not modulated by YB-1, confirming that YB-1 repression of the p53 promoter were specific. These results indicated that depletion of YB-1 levels induces apoptosis via transcriptional activation of p53. This was supported by the observation that, following depletion of YB-1 in SK-Mel-5 cells, levels of p53 were highly elevated as early as 2 hours post transfection.

Transcriptional regulation of p53 by YB-1 was further examined as follows. Full length p53 and Xenopus laevis elongation factor-1 (Xef-1)-capped RNA was prepared using the mMessage mMachine™ kit (Ambion Inc., Austin Tex.) according to the manufacturer's instructions, with linear DNA encoding each of these proteins fused to a T3 promoter. Translation reactions were performed using the Flexi® Rabbit Reticulocyte Lysate System (Promega, Madison Wis.). In a 10 μl final reaction volume, 0.05 μg RNA were mixed with 6.6 μl rabbit reticulocyte lysate, 0.2 μl amino acids minus methionine, 20 mM KCl, 0.1 μl RNAsin (XX) and 0.4 μl ³⁵S-Met, with either 4 μl SW buffer (12% glycerol, 4 mM Tris pH 7.9, 1 mM DTT, 100 mM KCl, 12 mM Hepes KOH pH 7.9, 1 mM EDTA, 40 mM NaCl) or increasing amounts of bacterially-expressed and purified GST-YB1 (200 ng/μl) in SW buffer, such that the final volume of SW buffer remained constant at 4 μl. After 60 min incubation at 30 ° C., an equal volume of 2× SDS-PAGE loading buffer was added and half of the sample was subjected to electrophoresis through a 10% SDS-PAGE gel. This was dried and translation products were visualized by autoradiography at room temperature.

The p53 RNA was translated efficiently producing a protein of 53 kDa in size. However, in the presence of increasing amounts of bacterially-expressed GST-YB1 protein, translation was significantly inhibited in a dose-dependent manner. The addition of 400 ng GST-YB1 reduced the amount of p53 protein product approximately 2 fold, while the addition of 800 ng GST-YB1 almost completely inhibits translation. The control Xef-1 RNA was efficiently translated to a protein of 50 kDa in size. The addition of up to 800 ng GST-YB1 had a minor inhibitory effect on Xef-1 translation. These results indicate that YB-1 is able to act post-transcriptionally to specifically inhibit the translation of p53.

Taken together, the above results indicate that reduction of the levels of free YB-1 within a population of cells will both increase p53 mRNA levels by preventing binding of YB-1 to the p53 promoter and increase p53 protein levels by relief of translational inhibition by YB-1.

### EXAMPLE 5

### The Role of YB-1 in Protecting Against Chemotherapeutic Agents and Agents that Induce DNA Damage

Many agents, for example chemicals like cisplatin, are used therapeutically to eradicate tumor cells by causing damage to DNA through induction of intra- or inter-strand crosslinks, leading to apoptosis of the tumor cells. This drug-induced apoptosis pathway is believed to occur via the activation of p53 (Muller et al., J. Exp. Med. 188:2033-2045, 1998). YB-1 has previously been shown to bind preferentially to damaged DNA (Ise et al., Cancer Res. 59:342-346, 1999). The following example shows that expression levels of YB-1 repressed genes are increased following treatment of human SK Mel-5 melanoma cells with the DNA-damaging drug, cisplatin. These results are consistent with the hypothesis that the binding of YB-1 to damaged DNA relieves the repression of YB-1 target genes.

SK Mel-5 cells were seeded at a density of 1×10⁶ cells/plate in 6 cm dishes 24 hours prior to treatment. Cisplatin was added to the cells at 300 ng/ml, which were then incubated at 37° C. and harvested at 2, 4, 6, 14 and 24 hours after treatment. Cells were harvested by scraping into PBS, washed by centrifugation and resuspended in RIPA buffer (50 mM Tris.HCl pH7.4, 1% NP40, 0.25% sodium deoxycholate, 150 mM NaCl, 1 mM PMSF, 1 μg/ml pepstatin, 1 μg/ml leupeptin, 1 μg/ml aprotinin). Protein was isolated from the soluble fraction and quantified by BCA assay (BioRad, Hercules, Calif.). Twenty μg of each protein sample, including an untreated SK Mel-5 cell control sample, was loaded on a 12% SDS-PAGE gel and electrophoresed at 135 V. The proteins were transferred to nitrocellulose in a Tris/glycine buffer. Proteins were detected using C-19 anti-p53 antibody (SC 1311; Santa Cruz Biotechnology Inc, Santa Cruz, Calif.), FL.261 anti-PCNA antibody (SC 7907; Santa Cruz Biotechnology Inc.) and M2 anti-Fas (anti-CD95) antibody (obtained from Immunex Corporation, Seattle, Wash.) and standard chemiluminescence detection protocols (Tropix, Foster City, Calif.)

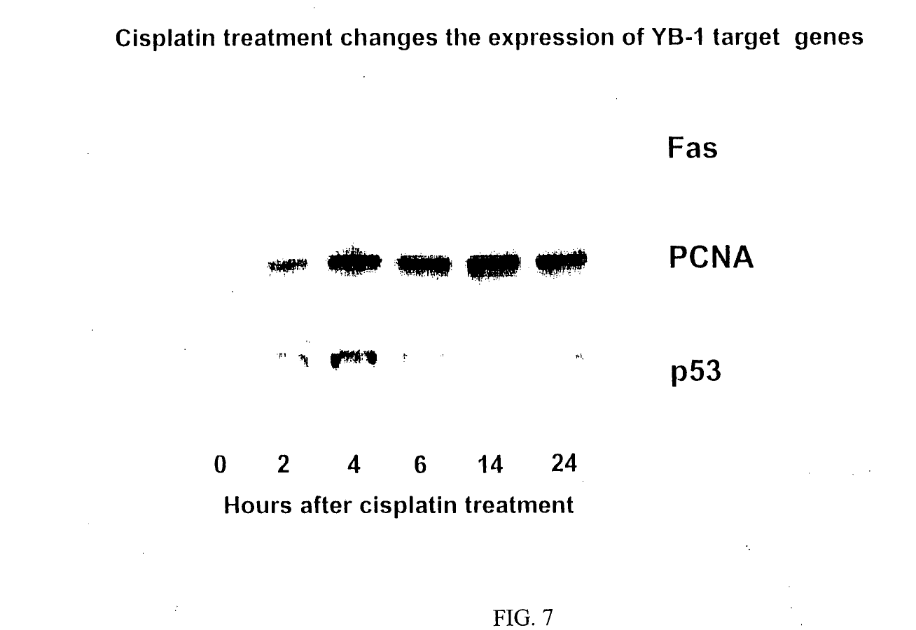

The expression profile of three YB-1 regulated genes (fas (CD95), p53 and PCNA (proliferating cell nuclear antigen)) after cisplatin damage to SK Mel 5 melanoma DNA is shown in the Western blot in FIG. 7. These three genes are normally repressed by YB-1, but following DNA damage, the expression levels of fas, p53 and PCNA increased. In this experiment, YB-1 levels remained unchanged (not shown), indicating that the increased expression levels of fas, p53 and PCNA were not due to decreased YB-1 levels.

### EXAMPLE 6

### Induction of Apoptosis in Tumor Cells Treated with Increasing Amounts of Cisplatin-Damaged DNA

SK Mel 5 melanoma cells were transfected with increasing amounts of pcDNA3 plasmid DNA treated with cisplatin to determine whether the damaged DNA acts as a YB-1 binding site, thereby leading to induction of apoptosis of these tumor cells. Untreated plasmid DNA (pcDNA3, Invitrogen, Carlsbad, Calif.) was used as negative control.

Plasmid pcDNA3 DNA was treated with cisplatin at a molar ratio of 35,000:1. 10 μg DNA was treated with 600 ng/ml cisplatin in a total reaction volume of 250 μl containing 25 μl buffer (30 mM NaCl, 5 mM sodium phosphate buffer) and 170 μl water. The reactions were incubated overnight at 37° C. and the excess cisplatin removed by ethanol precipitation. The pellet was resuspended in 20 μl water.

Transfection of SK Mel-5 Cells with Cisplatin Damaged DNA

SK Mel-5 cells were seeded at 1×10⁵ cells/well in 6 cm wells 24 hours prior to transfection. The cells were transfected with 0, 0.25, 0.5 and 1 μg cisplatin damaged DNA using Lipofectin 2000 and Optimem (Gibco BRL, Gaithersburg, Md.) according to the manufacturer's protocol. Control cells received 0, 0.25, 0.5 and 1 μg untreated DNA. After transfection, the cells were incubated at 37° C. for 4 hours, the medium replaced with complete RPMI containing 10% fetal calf serum and the cells incubated for 48 hours in an atmosphere containing 5% CO₂. Cells were harvested and counted after staining with trypan blue.

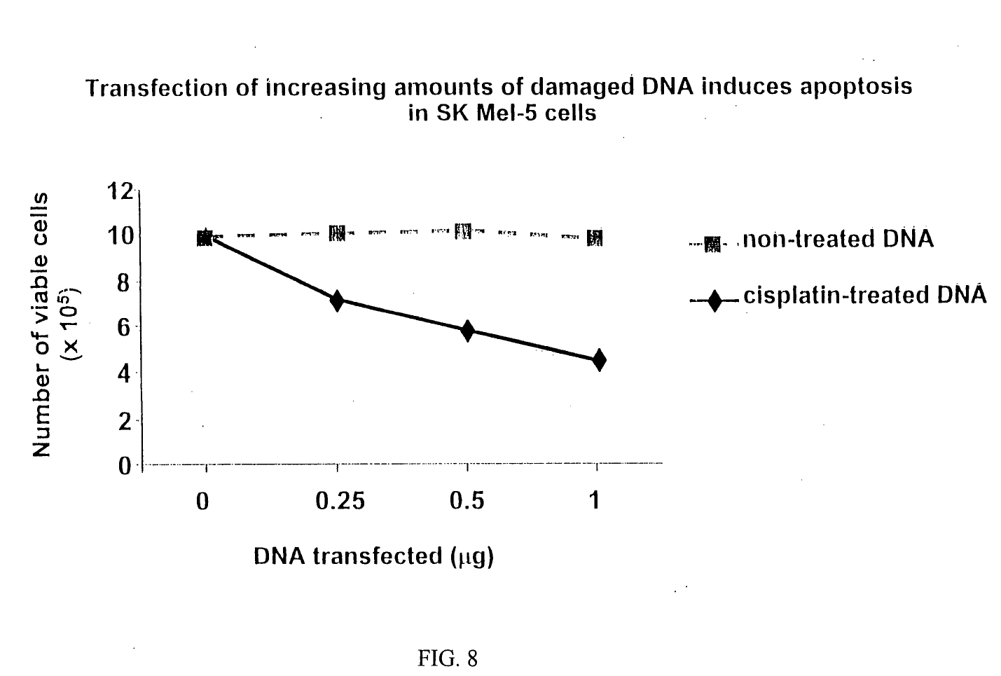

As shown in FIG. 8, results from this experiment demonstrate that the number of surviving cells decreased with increasing amounts of damaged DNA compared to cells that received untreated DNA. Transfection with undamaged DNA had no effect on live cell numbers.

### EXAMPLE 7

### Induction of Apoptosis in Tumor Cells Treated with Increasing Amounts of Cisplatin

As discussed above, transfection of a YB-1 binding site, or decoy, oligonucleotide into tumor cells induces apoptosis of these cells. SK-Mel-5 melanoma cells were transfected with pcDNA3 plasmid DNA treated with increasing amounts of cisplatin to determine whether the damaged DNA acts as a YB-1 binding site, leading to induction of apoptosis of these tumor cells. Untreated plasmid DNA (pcDNA3) was used as negative control.

Treatment of Plasmid DNA with Cisplatin.

The treatment of plasmid pcDNA3 DNA with different amounts of cisplatin was carried out according to the reactions shown in Table III below.

TABLE III

| | Molar ratio: | | | | |
|---|---|---|---|---|---|
| | 35000:1 | 3000:1 | 300:1 | 30:1 | 3:1 |
| Dilution of | — | — | 1/10 | 1/100 | 1/1000 |
| cisplatin | | | | | |
| Cisplatin | 50 μl | 4.2 μl | 4.2 μl of 1/10 | 4.2 μl of | 4.2 μl of |
| (600 ng/ul) | | | dilution | 1/100 dilution | 1/1,000 dilution |
| 10 ug DNA | 5 μl | 5 μl | 5 μl | 5 μl | 5 μl |
| 25 ul Buffer | 25 μl | 25 μl | 25 μl | 25 μl | 25 μl |
| Water | 170 μl | 215.8 μl | 215.8 μl | 215.8 μl | 215.8 μl |

The reactions were incubated overnight at 37° C. and the excess cisplatin removed by ethanol precipitation. The pellet was resuspended in 20 μl water.
Transfection of SK Mel-5 Cells with Cisplatin Damaged DNA

SK Mel-5 cells were seeded at 1×10⁵ cells/well in 6 cm wells 24 hours prior to transfection. The cells were transfected with 1 μg DNA using Lipofectin 2000 and Optimem (Gibco BRL, Gaithersburg, Md.) according to the manufacturer's protocol. After transfection, the cells were incubated at 37° C. for 4 hours, the medium replaced with complete RPMI containing 10% fetal calf serum and the cells were incubated for 48 hours in an atmosphere containing 5% CO₂. Cells were harvested and counted after staining with trypan blue.

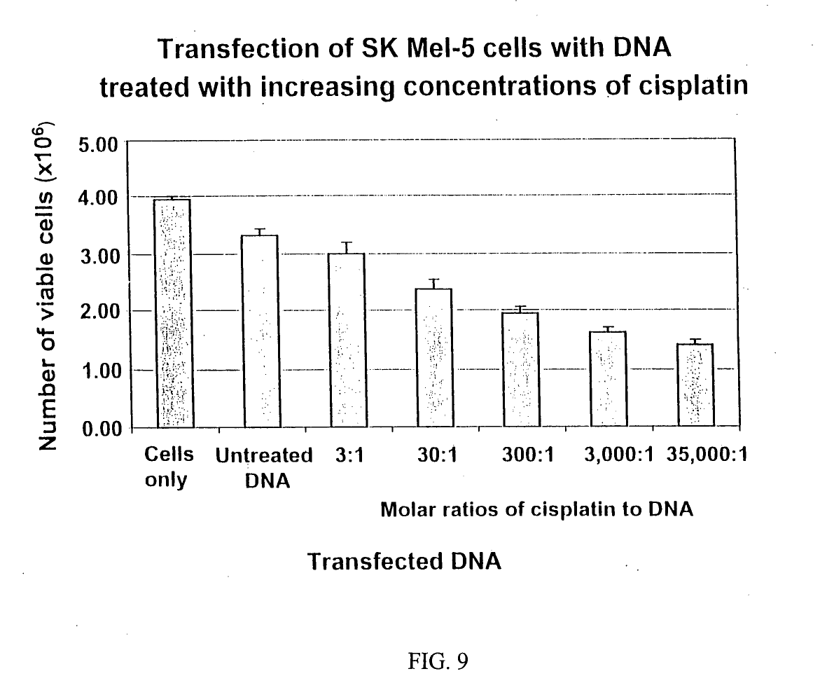

Results from this experiment (shown in FIG. 9) demonstrate that transfection with DNA treated with increasing concentrations of cisplatin resulted in decreased cell viability. Transfection with undamaged DNA had no effect on live cell numbers.

Taken together, these results indicate that drug-induced apoptosis may occur via the dissociation of YB-1 from the p53 promoter, leading to p53 expression and apoptosis.

### EXAMPLE 8

### Transfection of THP1 Cells with Cold Shock Proteins

THP1 cells are derived from human macrophages and endogenously express MHC Class II. The effect of bacterial cold shock proteins on the expression of MHC Class II in THP1 cells was examined as follows.

YB-1 was employed as a positive control since it is know to repress MHC Class II expression. Cells were co-transfected with the marker pGreen in order to identify which cells have been transfected. 2×10⁶ THP1 cells were transfected with 2 μg of each DNA as follows:

(a) pcDNA3 empty vector and pGreen;

(b) Mycobacterium bovis CSP in pcDNA3 and pGreen;

(c) Mycobacterium tuberculosis CSP in pcDNA3 and pGreen; and

(d) Rat YB-1 in pcDNA3 and pGreen.

All transfections were performed in triplicate. Cells were incubated with DNA for 5 hours before addition of 1 ml of cRMPI+20% FBS. After overnight incubation in a 5% CO₂ incubator, cells were transferred to 6 cm dishes with 10 ml cRMPI+5% FBS+50 ng/ml PMA. The cells were then allowed to grow for 3 days in a 5% CO₂ incubator. All transfections were divided into 2 tubes and stained with either 20 μg/ml of a pan MHC Class II antibody conjugated to phycoerythrin, or 20 μg/ml mouse IgG2a antibody conjugated to phycoerythrin for 1 hour on ice. Cells were sorted using a flow cytometer, gating on cells which were pGreen positive. Those cells expressing MHC Class II were identified as phycoerythrin positive.

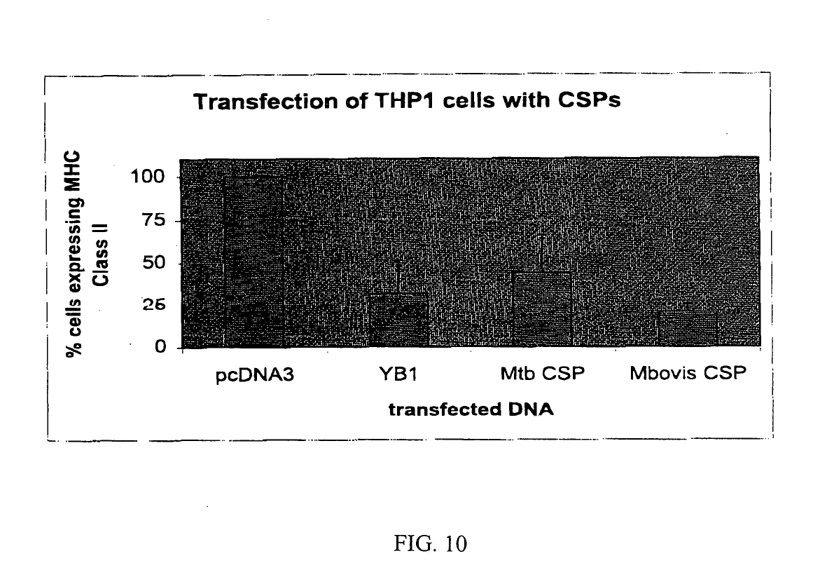

As shown in FIG. 10, a significant percentage of cells transfected with either YB-1 or the CSPs completely lost MHC Class II expression. These results indicate that the two bacterial cold shock proteins were able to affect expression of a host cell gene which assists the host cell in mounting an immune response within the body, and that intracellular pathogens are able to affect host cell immune response via their cold shock proteins.

### EXAMPLE 9

### Expression of a Protein by Toxoplasma gondii that Binds to the YB-1 Binding Site of the Human CD95 Promoter

Toxoplasma gondii proteins were prepared and tested in EMSA as follows.

Toxoplasma gondii (T. gondii) were cultured in neonatal human foreskin fibroblasts (NFF) as host cells. T. gondii proteins were isolated as follows. Media containing infected cells and T. gondii was centrifuged at 900×g for 15 mins and the supernatant removed. The pellet containing T. gondii was purified from NFF cellular debris by non-ionic chromatography through a cellulose column. The column eluate was collected, centrifuged as above, resuspended in SW buffer and frozen at −80° C. In parallel, uninfected NFF cells were lysed by successive syringing through a 27G needle. The extracts from the lysed cells were processed by non-ionic chromatography, centrifugation and storage as described above. The uninfected cell lysates were used as a control.

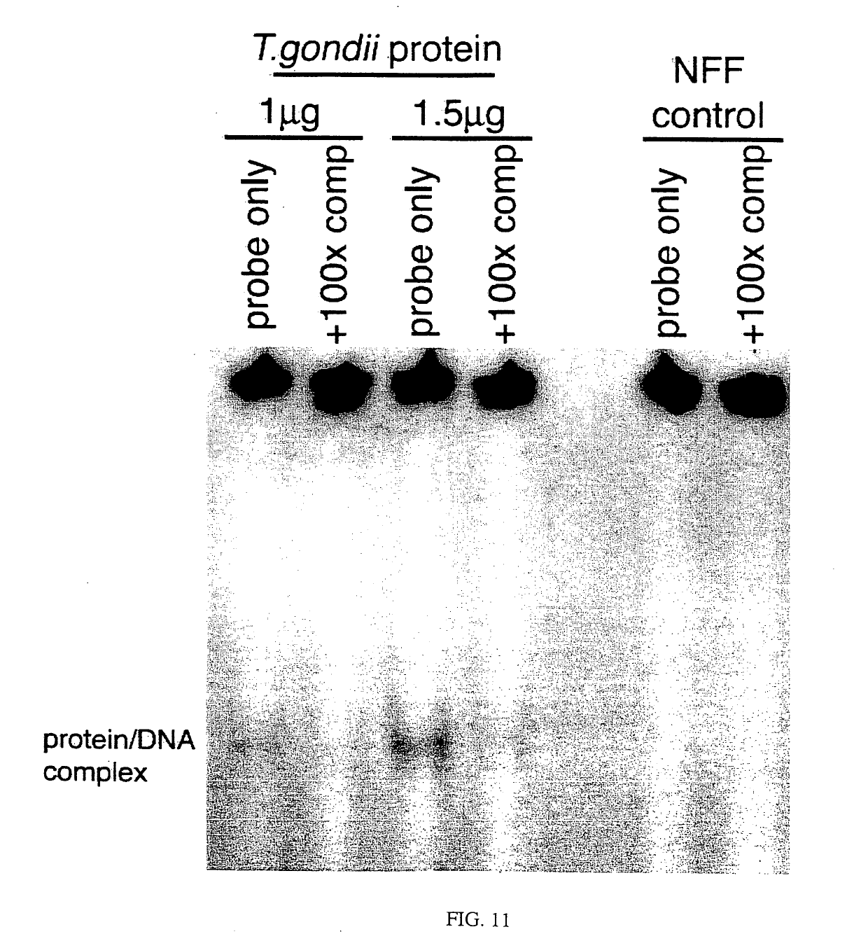

6.5 μl and 10 μl (equivalent to 1 μg and 1.5 μg, respectively) of T. gondii proteins were tested in an EMSA with the Fas silencer region (SEQ ID NO: 2) as probe. 10 μl of NFF control lysates were used as control. Reactions were set up in the absence and presence of 100× excess of an oligonucleotide previously shown to compete out YB-1 binding (SEQ ID NO: 9), and were performed as described above. Dried gel was exposed to film at −80° C. for 4 days. As shown in FIG. 11, protein/DNA complexes were formed in the presence of the probe of SEQ ID NO: 2, but not in the presence of the oligonucleotide of SEQ ID NO: 9 or with the control lysates.

All references and other materials cited herein are incorporated by reference in their entirety. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

We claim:

1. A method for modulating apoptotic cell death in a population of cells, comprising modulating the amount of a transcriptional regulator of apoptosis available to bind to a target polynucleotide in the cells, wherein the transcriptional regulator of apoptosis is a member of the Y-box nucleic acid binding family of polypeptides.

2. The method of claim 1, wherein the transcriptional regulator of apoptosis comprises an amino acid sequence selected from the group consisting of:
(a) SEQ ID NO: 39;
(b) sequences having at least 75% identity to SEQ ID NO: 39;
(c) sequences having at least 90% identity to SEQ ID NO: 39; and
(d) sequences that hybridize to SEQ ID NO: 39 under stringent conditions.

3. The method of claim 1, wherein the transcriptional regulator of apoptosis comprises an amino acid sequence selected from the group consisting of:
(a) SEQ ID NO: 40;
(b) sequences having at least 75% identity to SEQ ID NO: 40;
(c) sequences having at least 90% identity to SEQ ID NO: 40; and
(d) sequences that hybridize to SEQ ID NO: 40 under stringent conditions.

4. The method of claim 1, comprising contacting the population of cells with a genetic construct comprising a polynucleotide encoding a polypeptide selected from the group consisting of:
(a) human YB-1 (SEQ ID NO: 40); and
(b) the cold shock domain in human YB-1 (SEQ ID NO: 39);
(c) sequences having at least 75% identity to a sequence of SEQ ID NO: 39 or 40;
(d) sequences having at least 90% identity to a sequence of SEQ ID NO: 39 or 40;
(e) sequences that hybridize to a sequence of SEQ ID NO: 39 or 40 under stringent conditions.

5. The method of claim 1, wherein the cells are selected from the group consisting of: tumor cells; cells of the immune system; embryonic cells; cells of the nervous system; and cells infected with intracellular pathogens.

6. A method for increasing apoptotic cell death in a population of cells, comprising reducing the amount of a transcriptional regulator of apoptosis available to bind to a target polynucleotide in the cells, wherein the transcriptional regulator of apoptosis is a member of the Y-box nucleic acid binding family of polypeptides.

7. The method of claim 6, wherein the transcriptional regulator of apoptosis comprises an amino acid sequence selected from the group consisting of:
(a) SEQ ID NO: 39;
(b) sequences having at least 75% identity to SEQ ID NO: 39;
(c) sequences having at least 90% identity to SEQ ID NO: 39; and
(d) sequences that hybridize to SEQ ID NO: 39 under stringent conditions.

8. The method of claim 6, wherein the transcriptional regulator of apoptosis comprises an amino acid sequence selected from the group consisting of:
(a) SEQ ID NO: 40;
(b) sequences having at least 75% identity to SEQ ID NO: 40; and
(c) sequences having at least 90% identity to SEQ ID NO: 40; and
(d) sequences that hybridize to SEQ ID NO: 40 under stringent conditions.

9. The method of claim 6, wherein the cells are tumor cells.

10. The method of claim 6, comprising contacting the population of cells with an anti-sense oligonucleotide directed against the transcriptional regulator of apoptosis.

11. The method of claim 10, wherein the anti-sense oligonucleotide comprises a sequence selected from the group consisting of: SEQ ID NO: 19-23 and 29-33.

12. The method of claim 6, comprising contacting the population of cells with a decoy oligonucleotide comprising a transcriptional regulator of apoptosis binding site

13. The method of claim 12, wherein the decoy oligonucleotide comprises a sequence selected from the group consisting of: SEQ ID NO: 2, 9-15 and 17.

14. A method for modulating apoptotic cell death in a population of cells, comprising modulating the binding of a transcriptional regulator of apoptosis to a regulatory polynucleotide in the cells, wherein the transcriptional regulator of apoptosis is selected from the group consisting of:
(a) members of the Y-box nucleic acid binding family of polypeptides;
(b) SEQ ID NO: 39 and 40;
(c) sequences having at least 75% identity to a sequence of SEQ ID NO: 39 or 40; and
(d) sequence having at least 90% identity to a sequence of SEQ ID NO: 39 or 40; and
(e) sequences that hybridize to a sequence of SEQ ID NO: 39 or 40 under stringent conditions.

15. A method for increasing the sensitivity of tumor cells to a DNA-damaging agent, comprising contacting the tumor cells with an oligonucleotide selected from the group consisting of:
(a) decoy oligonucleotides comprising a transcriptional regulator of apoptosis binding site; and
(b) anti-sense oligonucleotides directed against a transcriptional regulator of apoptosis;
wherein the transcriptional regulator of apoptosis is a member of the Y-box nucleic acid binding family of polypeptides.

16. A method for increasing p53-mediated apoptosis in a cell population, comprising reducing the amount of a transcriptional regulator of apoptosis available to bind to a target polynucleotide in the cells and thereby increasing expression of p53, wherein the transcriptional regulator of apoptosis is a member of the Y-box nucleic acid binding family of polypeptides.

17. The method of claim 16, wherein the transcriptional regulator of apoptosis is selected from the group consisting of:
(a) SEQ ID NO: 39;
(b) sequences having at least 75% identity to SEQ ID NO: 39;
(c) sequences having at least 90% identity to SEQ ID NO: 39; and
(d) sequences that hybridize to SEQ ID NO: 39 under stringent conditions.

18. The method of claim 16, wherein the transcriptional regulator of apoptosis is selected from the group consisting of:
(a) SEQ ID NO: 40;
(b) sequences having at least 75% identity to SEQ ID NO: 40;
(c) sequences having at least 90% identity to SEQ ID NO: 40; and
(d) sequences that hybridize to SEQ ID NO: 40 under stringent conditions.

19. A method for increasing sensitivity to apoptosis in a population of cells harboring intracellular pathogens, comprising reducing the amount of a cold shock protein available to bind to a target polynucleotide in the cells.

20. A method of stimulating an immune response against an intracellular pathogen in a population of cells harboring the intracellular pathogen, comprising reducing the amount of a cold shock protein available to bind to a target polynucleotide in the cells.
